(19) United States
(12) Patent Application Publication
(10) Pub. No.: US 2016/0244510 A1
(43) Pub. Date: Aug. 25, 2016

(54) HUMAN MONOCLONAL ANTIBODIES

(71) Applicant: DUKE UNIVERSITY, Durham, NC (US)

(72) Inventors: M Anthony Moody, Durham, NC (US); Hua-Xin Liao, Durham, NC (US); Barton F Haynes, Durham, NC (US); Lynn Morris, Johannesburg (ZA)

(21) Appl. No.: 15/025,044

(22) PCT Filed: Sep. 26, 2014
(86) PCT No.: PCT/US2014/057743

§ 371 Date: Mar. 25, 2016

Related U.S. Application Data
(60) Provisional application No. 61/883,220, filed on Sep. 27, 2013.

(51) Int. Cl. C07K 16/10; A61K 9/06; A61K 9/00

(52) U.S. Cl. CPC C07K 16/1045; A61K 9/0019; A61K 9/06; C07K 2317/76; C07K 2317/732

(57) ABSTRACT

The present invention relates, in general, to HIV-1-reactive antibodies and, in at least certain specific embodiments, to broadly neutralizing antibodies (bnAbs) (and fragments and derivatives thereof) and to compositions comprising same. The invention further relates to methods of using such bnAbs (and fragments and derivatives thereof) and compositions in immunotherapy regimens (e.g., passive immunotherapy regimens). The antibodies (and fragments and derivatives thereof) disclosed herein can also be used in methods of identifying candidate immunogens for use in inducing an immune response against HIV-1 in a mammal (e.g., a human). The invention also relates to such methods and to immunogens so identified.

This application claims the benefit of U.S. Application Ser. No. 61/883,220 filed Sep. 27, 2013, the entire contents of which application are hereby incorporated by reference

This invention was made with government support under Grant Nos. U19 AI067854 and UM1 AI100645 awarded by the National Institutes of Health. The government has certain rights in the invention.

### TECHNICAL FIELD

The present invention relates, in general, to HIV-1-reactive antibodies and, in at least certain specific embodiments, to broadly neutralizing antibodies (bnAbs) (and fragments and derivatives thereof) and to compositions comprising same. The invention further relates to methods of using such bnAbs (and fragments and derivatives thereof) and compositions in immunotherapy regimens (e.g., passive immunotherapy regimens). The antibodies (and fragments and derivatives thereof) disclosed herein can also be used in methods of identifying candidate immunogens for use in inducing an immune response against HIV-1 in a mammal (e.g., a human). The invention also relates to such methods and to immunogens so identified.

### BACKGROUND

Induction of antibodies with neutralization breadth is a primary goal of HIV-1 vaccine development (Karlsson Hedestam et al, Nat. Rev. Microbiol 6(2):143-155 (2008). Broadly neutralizing antibodies (bnAbs) have been demonstrated to protect against acute infection in animal models (Mascola et al, Nat. Med. 6(2):207-210 (2000), Hessell et al, J. Virol. 84(3):1302-1313 (2010)), and, since 2009, a large number of new monoclonal antibodies have been isolated (Burton et al, Science 337(6091):183-186 (2012), Bonsignori et al, Trends Microbiol. 20(11):532-539 (2012), Mascola and Haynes, Immunol. Rev. 254(1):225-244 (2013)), providing new strategies for vaccine design aimed at eliciting those antibodies (Haynes et al, Nat. Biotechnol. 30(5):423-433 (2012)). It has been shown that bnAbs only arise after several years of HIV-1 infection (Tomaras et al, J. Virol. 82(24):12449-12463 (2008), Gray et al, J. Virol. 85(10):4828-4840 (2011)). Studies of bnAbs given to chronically (Armbruster et al, AIDS 16(2):227-233 (2002)) or acutely (Mehandru et al, J. Virol. 81(20):11016-11031 (2007)) HIV-1 infected subjects showed little impact on the course of infection, suggesting that the presence of bnAbs alone in most individuals with established infection cannot prevent disease progression

All current HIV-1 envelope (Env) immunogens induce a narrow neutralizing antibody response that in standard TZM-bl pseudovirus neutralization assays predominantly inhibit tier 1 HIV-1 Env pseudoviruses (Montefiori et al, J. Infect. Dis. 206(3):431-441 (2012)). Thus, a critical question for vaccine development is whether easily induced, narrow neutralizing antibodies can mediate immune pressure sufficient to select for virus escape mutants.

The ALVAC/AIDSVAX B/E vaccine used in the RV144 vaccine efficacy trial in Thailand induced an estimated vaccine efficacy of 31.2%; however, antibodies induced by this vaccine were capable of neutralizing only tier 1 laboratory-adapted HIV-1 strains but not tier 2 strains across HIV-1 clades (Montefiori et al, J. Infect. Dis. 206(3):431-441 (2012), Liao et al, Immunity 38(1):176-186 (2013)). Interestingly, RV144-induced antibodies directed to the first and second variable (V1V2) region of Env gp120 correlated with decreased infection risk (Haynes et al, N. Engl. J. Med. 366(14):1275-1286 (2012)), and V2 antibodies isolated from vaccinees, while not capturing nor neutralizing HIV-1 primary strain virions, did bind to the surface of primary virus-infected cells and mediated antibody-dependent cellular cytotoxicity (ADCC) Liao et al, Immunity 38(1):176-186 (2013)). A study of virus sequences from breakthrough infections in RV144 participants showed vaccine-induced immune pressure at amino acid position 169 in V2 of Env gp120 (Rolland et al, Nature 490(7420):417-420 (2012)). These data are consistent with the hypothesis that the estimated 31.2% protection in RV144 was mediated by antibodies targeted at the V2 region but, at present, it is not known whether tier 1 neutralization or ADCC effector functions were responsible.

Determining whether an in vitro assay is a surrogate for in vivo protection against HIV-1 is difficult and requires passive protection studies of rhesus macaques challenged with simian-human immunodeficiency virus (SHIV) (Mascola et al, Nat. Med. 6(2):207-210 (2000), Moldt et al, Proc. Natl. Acad. Sci. USA 109(46):18921-18925 (2012), Girard and Plotkin, Curr. Opin. HIV AIDS 7(1):2012)) or demonstration that a particular immune response can select for virus escape mutants in vivo (Goonetilleke et al, J. Exp. Med. 206(6):1253-1272 (2009), Miura et al, J. Viro. 83(6):2743-2755 (2009)). In order to determine whether tier 1 neutralizing antibodies are capable of exerting immune pressure, a chronically HIV-1 clade C infected Tanzanian individual (CH0457) has been studied over two years and both tier 1 (narrow) and tier 2 (broad) neutralizing antibodies have been isolated from the same individual. In addition, a large panel of full-length env genes from multiple time points was isolated from CH0457 that were used to generate autologous pseudoviruses for testing for antibody-mediated virus neutralization and evidence of antibody-mediated immune pressure. The present invention results, at least in part, from studies demonstrating that bnAbs with the ability to broadly neutralize tier 2 viruses exerted profound immune pressure (94% escape mutants) on the autologous virus quasispecies. In contrast, a clonal lineage of CD4 binding site (CD4bs) narrow neutralizing antibodies with neutralizing activity only against tier 1 viruses exerted minimal autologous virus immune pressure (13% escape mutants) during the time studied. These data suggest that HIV-1 Env tier 1 neutralizing antibodies will not be able to prevent HIV-1 transmission by virion neutralization.

### SUMMARY OF THE INVENTION

In general, the present invention relates to HIV-1-reactive antibodies. In at least certain specific embodiments, the invention relates to bnAbs (and fragments and derivatives thereof) and to compositions comprising same. The invention further relates to methods of using such bnAbs (and fragments and derivatives thereof) and compositions in immunotherapy regimens (e.g., passive immunotherapy regimens). The invention also relates to methods of using such bnABs (and fragments and derivatives thereof) to identify candidate immunogens that can induce an immune response against HIV-1 in a mammal (e.g., a human), and to immunogens so identified.

Certain objects and advantages of the present invention will be clear from the description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Clonal lineages derived from participant 0457. A: PBMC were stained with a panel of antibodies to identify B-cell-specific markers, non-B-cell markers, and with antigen-specific reagents (gp120_{ConC}). Cells shown are memory B cells; the kite-shaped gate was sorted as single cells into 96-well plates, with a diagonal of gp120_{ConC} core+/+ isolated. The frequency of antigen-specific cells was similar in both sorted samples, representative data from the week 8 sample shown. B: Two IgG1 gp120 V3 mAbs (CH14, CH48) were isolated and were not related to other isolated mAbs. Clonal lineage CH13 consisted of six IgG1 mAbs that used V_{H}1˜69*01/J_{H}3*02 and V_{K}1˜39*01/J_{K}4*01, and had a mean heavy chain mutation frequency of 9.8%. Lineage CH27 consisted of three mAbs, two IgA2 (CH27, CH28) and one IgG1 (CH44); this lineage used V_{H}3˜66*02/J_{H}2*01 and V_{K}3˜20*01/J_{K}1*01, and had a mean heavy chain mutation frequency of 15.7%. All trees are plotted on the same scale. C: Clonal lineage CH13 mAbs were tested for sensitivity to amino acid substitution in binding and neutralization assays (Tables S2 and S3) Residues found to be critical for mAb binding are highlighted in the crystal structure of gp120 C.YU2 complexed with mAb 17b and CD4 (72). Antibody 17b removed for clarity; CD4 is shown in light gray and gp120 in light blue. Mapped residues are largely located within the CD4-gp120 contact surface. Residues in the V1/V2 loop are not shown; the gp120 used for this crystal structure lacked that feature. D. Antibodies CH14 and CH48 were tested for binding to an array of peptides reflective of multiple HIV-1 clades. Both antibodies bound to peptides reflective of the V3 loop (residues 301-325) across multiple clades; no binding was observed for other epitopes within gp120 or gp41.

FIGS. 2A and 2B. Heterologous neutralization by mAbs from participant CH0457. Antibodies were tested against a panel of tier 1 (2A) and tier 2 (2B) viruses from diverse clades. Antibodies with detectable neutralization are shown in colored boxes with the EC50 concentration. Control polyclonal antibody preparation HIVIG-C is shown to the right of the mAbs. Serum from participant 0457 at the week 8 and week 96 time points is shown on the right, also in colored boxes with the EC50 reciprocal dilution values. Lineage CH13 mAbs and the non-lineage mAbs CH14, CH15, and CH48 potently neutralized tier 1 viruses but only weakly neutralized a single tier 2 virus (C.246F_C1G). In contrast, lineage CH27 neutralized a single tier 1 virus but neutralized 23/40 (58%) of tier 2 viruses. Antibody HJ16 neutralization data include published reports (25, 73) and additional data. The participant serum neutralized all tier 1 viruses at >1:20, and 37/40 (93%) and 31/40 (78%) of tier 2 viruses at week 8 and week 96, respectively.

FIG. 3. Neutralization of heterologous viruses by mAbs from participant CH505. V3 loop mAbs DH151 and DH228 from participant CH505 were tested against a heterologous HIV isolate panel. Two of four tier 1 isolates were neutralized by the mAbs; none of the 16 tier 2 isolates were neutralized by the mAbs.

FIG. 4. Neutralization of mAbs against autologous viruses and Env sequence phylogenies. Data from CH0457 shown in A and B; data from CH505 shown in C and D. Neutralization by autologous serum and isolated mAbs shown as a heat map (A and C). A panel of 84 pseudoviruses amplified from participant CH0457 that spanned the study period was tested. Each row in the neutralization panel (A) and phylogeny tree (B) depicts a distinct Env isolate from longitudinal sampling, spanning week 0 (enrollment; red) to week 96 after enrollment (purple). Provirus sequences isolated from PBMC are also shown in grey. The phylogeny only shows those Envs for which neutralization data was obtained; the full phylogeny for CH0457 is in FIG. 7. Neutralization of autologous serum (reciprocal dilution) and isolated mAbs (concentration in μg/mL) shown. Antibody data (A) are shown for lineage CH13 mAbs (Tier 1 CD4bs), lineage CH27 mAbs (Tier 2 CD4bs), and CH14 and CH48 (Tier 1 V3). For CH505, neutralization data (C) and phylogeny (D) are shown; Env sequences span transmission (week 0, red) through week 100 (purple). Antibody data for DH151 and DH228 (Tier 1 V3) and lineage CH103 mAbs (Tier 2 CD4bs) are shown.

FIG. 5. Recognition of Env epitopes by antibodies without neutralization breadth. The dark blue region in the interior of the binding pocket represents conserved gp120 epitopes targeted by CD4bs or V3 mAbs. In CH0457 and CH505, these antibodies evolved to accommodate and bypass the variable gp120 regions on autologous viruses that potentially limit access to the epitope. This results in a good fit by autologous antibodies for Envs with low reactivity (ie, tier 1B or tier 2 virus Envs) (A). On heterologous tier 2, low-reactivity Envs (B), conformational change is resisted (34, 35), thus the antibodies fail to bind and neutralize. In contrast, on heterologous tier 1A viruses, Env reactivity is high, thus Env can undergo conformational change more readily (C). Therefore, even though the antibody surface complements only the epitope and not the surrounding variable gp120 structures, the variable structures are conformationally flexible on tier 1A and some tier 1B virus high-reactivity Envs, allowing the antibody to bind and neutralize.

FIG. 6. Cross blocking of HJ16 and lineage CH27 mAbs. Antibodies from lineage CH27 were tested for cross-blocking against HJ16. Taken together, the data suggest that the binding sites for the lineage CH27 mAbs and HJ16 overlap but are not identical. A. HJ16 was immobilized on a surface plasmon resonance chip and antibody-Env mixtures were flowed over the chip to determine if the antibody-Env complex bound to HJ16. Control mAb palivizumab was the control; non-neutralizing anti-HIV-1 mAb 16H3 did not significantly block binding to HJ16. In contrast, HJ16 blocked to 96% as expected, while CH27 and CH44 blocked about ⅓ of binding to HJ16. B. CH27 immoblized on a chip was able to bind to Env mixed with palivizumab or 16H3, but binding was partially blocked when Env was mixed with CH27, CH44, or HJ16. C. CH44 immoblized on a chip was able to bind to Env mixed with palivizumab or 16H3, but binding was blocked when Env was mixed with CH27, CH44, or HJ16.

FIG. 7. HIV-1 env gene evolution in participant CH0457. Env phylogeny from CH0457 during chronic infection is shown. A pixel map (left) depicts mutations where each site differs from the consensus of earliest plasma Envs, whether mutations (red) or insertions/deletions (black). Each row in the tree and the pixel map depicts a distinct Env isolated from longitudinal samples; i.e., week 0 (enrollment; red) through week 96 post-enrollment (purple). Env provirus sequenced from PBMCs in the enrollment sample are also shown (grey). The phylogeny was inferred from protein sequences by PhyML (5) with the HIVw substitution model (6). Node labels indicate at least 60% bootstrap support. Root placement was chosen to minimize the sum of variances among within-timepoint distances (7, 8). A group of six provirus-derived Envs was enriched for APOBEC3G hypermutations (4), as identified by a square bracket and asterisk. Neutralization titers (μg/mL) from two representative mAbs (CH14, CH16) are shown in two columns between the pixel map and the tree for the subset of Envs assayed. Locations of V1-V5 and other Env landmarks are shown by (faint grey boxes) and sites that contact CD4 are shown near the top of the pixel map (pink tic marks).

FIG. 8. Neutralization of autologous viruses from CH0457 by mAbs. A: Antibodies were tested against a panel of 84 pseudoviruses amplified from plasma from participant CH0457 that spanned the study period. Antibodies from lineage CH13 neutralized 52/84 (62%) of isolates tested and mAbs from this lineage were active against at least one isolate from each of the time points tested. For mAbs from lineage CH13, neutralization titers ranged from 0.8-50 g/mL. In contrast, mAbs from lineage CH27 neutralized only 5/84 (6%) of isolates; neutralization titers ranged from 44-50 μg/mL. Control mAbs are shown with asterisks above their names; narrow neutralizing CD4bs mAb F105 (9) weakly neutralized 2/72 (2.8%) while bnAb HJ16 (10) potently neutralized 5/72 (6.9%) of pseudoviruses. Anti-HIV-1 bnAbs CH31 (11) and CH106 (2) neutralized 73/84 (87%) and 55/62 (89%) respectively with titers ranging from <0.02 to 46 μg/mL, while anti-influenza bnAb CH65 (12) weakly neutralized a single isolate (w72.4). Testing of the autologous viruses by these and additional samples (FIG. 11) was used to classify the viruses for neutralization sensitivity (Tier Classification). B: HIV-1 Env sequences were amplified by single genome amplification from week 0 PBMC. Env sequences from plasma are indicated by a “p”; cell derived sequences are indicated by a “c”. Pseudoviruses made from these Env sequences were tested against the panel of mAbs isolated from CH0457. Of the 34 pseudoviruses tested, 28/34 (82%) were sensitive to the V3 mAbs CH14 and CH48 and 11/34 (32%) were sensitive to the CD4bs-directed lineage CH13 mAbs. Only 5/34 (15%) of pseudoviruses were sensitive to the nAb lineage CH27 mAbs; of these, the two Envs most distant in the phylogenetic tree from the week 0 plasma Envs, w0.29c and w0.35c, were the most sensitive to neutralization (IC50 range 0.1-2.0 μg/mL).

FIG. 9. Neutralization of autologous viruses from CH505 by mAbs. Antibodies DH151 and DH228 were tested against a panel of 96 autologous pseudoviruses from participant CH505. Tier 1 V3 mAbs neutralized 45/96 (47%, range 50-0.03 μg/mL) of the autologous viruses. Like CH0457 tier 1 V3 abs, mAbs DH151 and DH228 neutralized 7/96 (7.3%) viruses at ≦2 μg/mL. Testing of the autologous viruses against HIVIG-C and a panel of well characterized sera from clade C infected participants (SA-C8, SA-C36, SA-C82, SA-C102) (FIG. 13) was used to classify the viruses for neutralization sensitivity (Tier Classification).

FIG. 10. Autologous neutralization by serum from participant CH0457. Serum from participant CH0457 spanning the study period was tested against 84 autologous virus isolates from the same time period and two autologous viruses isolated from PBMC. Control HIVIG-C pooled antibodies are shown on the right. Serum antibodies from CH0457 neutralized autologous viruses from all early time points, and serum from weeks 48, 72, and 96 showed greater potency against autologous viruses. Virus isolates from week 96 were resistant to plasma from all time points, suggesting that a new escape event may have occurred during the later study period. Six viruses were tested for sensitivity to a panel of five well characterized serum samples; these viruses demonstrated an intermediate sensitivity to these sera, consistent with an intermediate phenotype (tier 1b). Companion data for these sera against other HIV-1 strains is shown in FIG. 12.

FIG. 11. Neutralization of mAbs against autologous viruses from CH0457: extended panel. Data shown here include some neutralization data shown in FIG. 4A and FIG. 8. Twenty of the viruses were tested against a panel of V3 and CD4bs mAbs with restricted neutralization profiles (13-19) and a panel of well-characterized HIV-1-infected patient serum samples. These neutralization profiles were used to classify the pseudoviruses for neutralization sensitivity.

FIG. 12. Neutralization of a panel of HIV-1 isolates by well characterized serum samples. Five HIV-1 isolates were tested against five well characterized serum samples. The canonical tier 1 virus MN.3 was very sensitive to the serum samples. The intermediate sensitive virus 6535.3 was more resistant than MN.3 but not as resistant as the three tier 2 viruses.

FIG. 13. Neutralization of mAbs against autologous viruses from CH505, tabular format. Data shown in FIG. 4C are here supplemented with additional neutralization data. Fifteen pseudoviruses were tested against a panel of mAbs and well characterized HIV-1-infected patient serum samples. Isolates that were sensitive to the autologous V3 mAbs DH151 and CH228 were also mostly sensitive to heterologous mAbs. Sensitivity to the mAbs and sera were used to refine the tier classification shown in the rightmost column

FIG. 14. Antibody sequences. Nucleotide sequences encoding the heavy chain (HC) and light (kappa) chain (KC) of monoclonal antibodies CH27, CH28 and CH44 are shown, as are the amino acid sequences. The underlined sequences correspond to CDR1, italicized to CDR2 and underlined and italicized to CDR3.

FIG. 15. Sequence signature. Analysis of all deposited group M Env sequences showed that 58% of the isolates had a glycosylation site at 130

FIG. 16. ADCC Activity of CH27, CH28 and CH44.

### DETAILED DESCRIPTION OF THE INVENTION

The global HIV/AIDS epidemic remains a global health threat. While there is not yet a cure, significant advances in the treatment of HIV-1 infection have occurred. A vaccine effective against the most common modes of transmission of the virus will likely need to induce antibodies that have the capacity to block infection by a wide array of possible viral targets and that can be present at mucosal surfaces (e.g., the lower gastrointestinal tract and genital tract).

One class of antibodies capable of blocking infection by a wide array of HIV-1 strains is bnAbs. Within the last five years, there have been a large number of new bnAbs isolated with a concomitant increase in the number of known epitope targets for bnAbs. These targets reflect relatively conserved epitopes on HIV-1 and have consisted of regions that mimic human antigens and are thereby under immune tolerance control (Yang et al, J. Exp. Med. 210(2):241-256 (2013)), post-translational modifications added by human cells (Pejchal et al, Science 334(6059)L1097-1103 (2011), Sanders et al, J. Virol. 76(14):7293-7305 (2002)), or epitopes that must be conserved to maintain functionality of the HIV-1 envelope protein (Sanders et al, J. Virol. 76(14):7293-7305 (2002)). A great deal of effort has been invested in developing vaccines that elicit such antibodies (Esparza, Vaccine 31(35):3502-3518 (2013)). The identification of additional targets for bnAbs remains a priority.

Antibody responses at mucosal surfaces consist of antibodies of the IgG and IgA classes. IgG antibodies are the predominant isotype found in plasma and can be actively or passively transported across anatomical barriers. IgA antibodies can also be found in plasma at lower concentrations but can also be locally produced and actively transported across mucosal barriers. IgA antibodies are particularly adapted for survival at mucosal surfaces; e.g., IgA2 antibodies are resistant to some bacterial proteases found in respiratory tract pathogens. It is expected that for protection against HIV-1 infection that would occur via mucosal surfaces (e.g., sexual transmission, breast milk transmission), IgA antibodies will be critically important.

A series of antibodies have been isolated from a chronically HIV-1-infected subject from Tanzania (CH0457). Using antigen-specific flow cytometry, B cells expressing HIV-1-reactive antibodies were isolated from this subject. Genes from these cells were isolated by overlapping PCR and antibodies expressed for screening. Based on the screening, a number of HIV-1-reactive antibodies were identified that were sent for neutralization assays; of these antibodies, three (CH27, CH28 and CH44) were found to be broadly neutralizing. Two of these three antibodies (CH27 and CH28) were of the IgA2 isotype. These are the first natural IgA bnAbs that have been isolated.

Mapping of this group of bnAbs revealed that they did not map to any known bnAb specificity. These data suggest that this group of bnAbs binds to a novel bnAb epitope. Combined with the fact that the isolated bnAbs were of the IgA isotype, this group of antibodies represent a target for vaccine development and represent a therapeutic for the prevention of mucosal HIV-1 transmission.

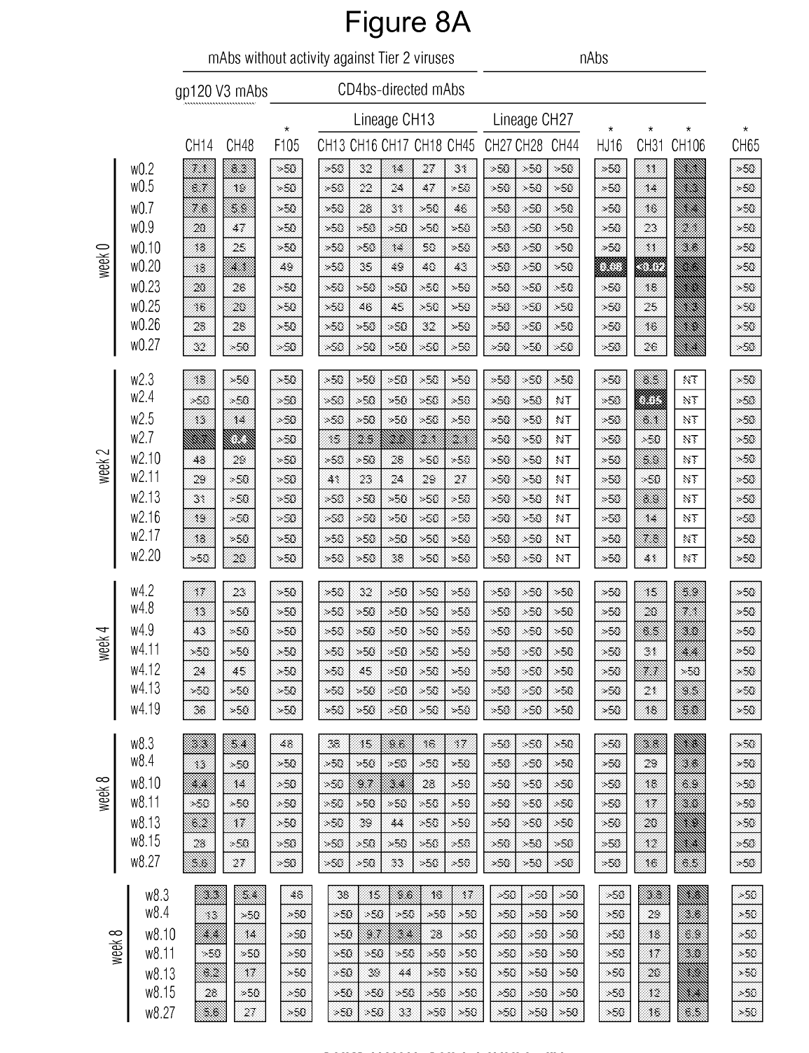

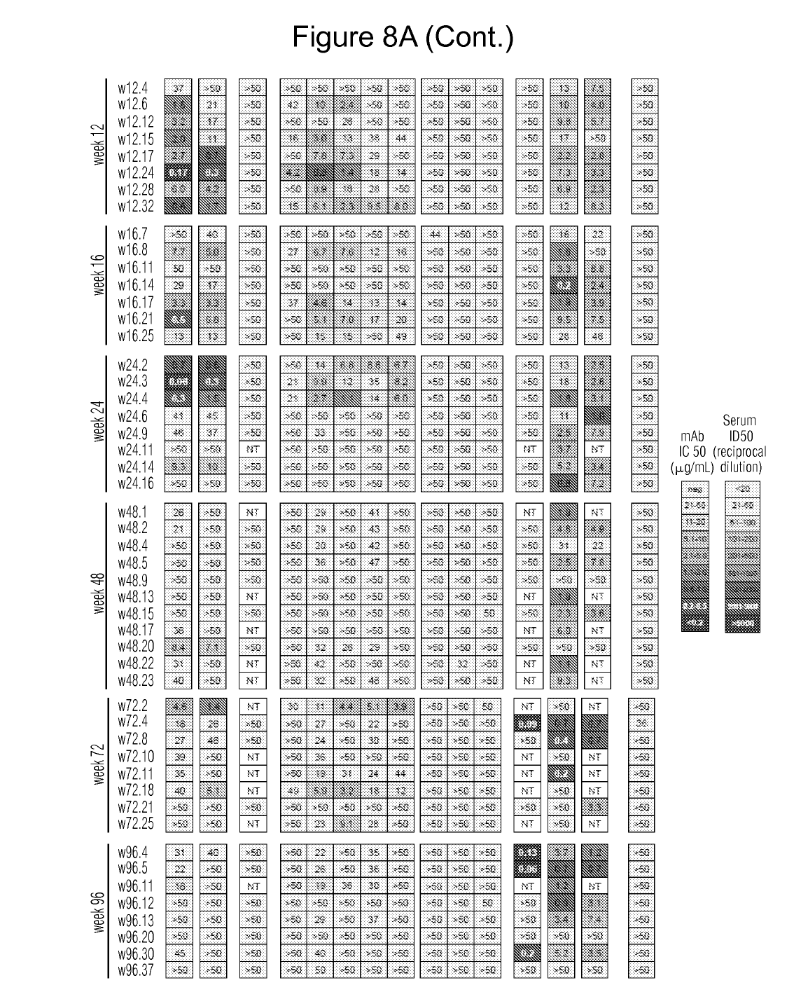

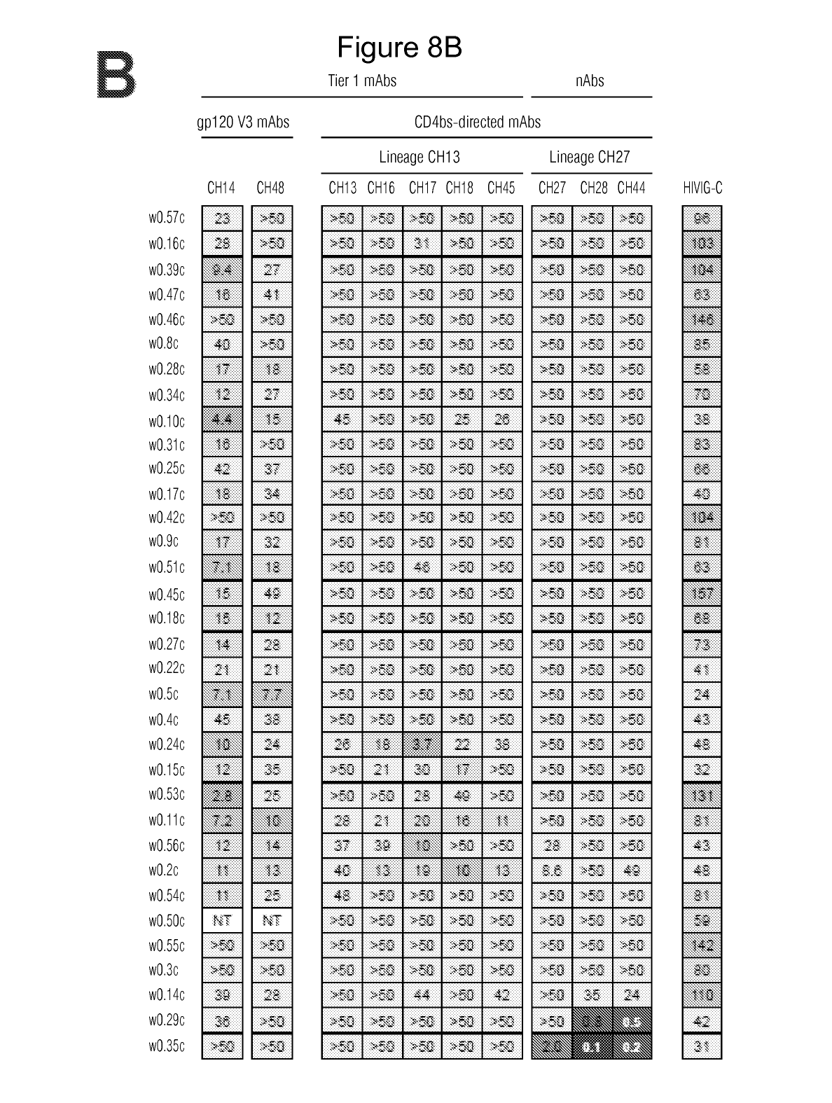

The present invention relates to the bnAbs disclosed herein (e.g., the IgA bnAbs), to antibodies having the specificity of the disclosed bnAbs, and to fragments (e.g., antigen-binding fragments) and derivatives thereof, and to methods of using same to inhibit HIV-1 infection in a subject (e.g., a human). The invention includes intact antibodies and fragments (e.g., Fab, Fab′, F(ab′)₂, FV, CDR (see FIG. 8)) thereof. The invention also includes nucleic acids comprising nucleotide sequences encoding such antibodies and fragments thereof (e.g., Fab, Fab′, F(ab′)₂, FV and CDR fragments), and to constructs (e.g., vectors) comprising same.

Preferred antibodies of the invention for therapeutic use include those comprising variable heavy (VH) and light (VL) chain amino acid sequences selected from those shown in FIG. 8. In accordance with the methods of the present invention, either intact antibody or fragment thereof (e.g., antigen binding fragment) can be used. That is, for example, intact antibody, a Fab fragment, a diabody, or a bispecific whole antibody can be used to inhibit HIV-1 infection in a subject (e.g., a human). Toxins can be bound to the antibodies or antibody fragments described herein. Such toxins include radioisotopes, biological toxins, boronated dendrimers, and immunoliposomes (Chow et al, Adv. Exp. Biol. Med. 746:121-41, 2012)). Toxins can be conjugated to the antibody or antibody fragment using methods well known in the art (Chow et al, Adv. Exp. Biol. Med. 746:121-41 (2012)). Combinations of the antibodies, or fragments or derivatives thereof, disclosed herein can also be used in the methods of the invention.

The antibodies, and fragments/derivatives thereof, described above can be formulated as a composition (e.g., a pharmaceutical composition). Suitable compositions can comprise the bnAb or fragment (or derivative thereof) dissolved or dispersed in a pharmaceutically acceptable carrier (e.g., an aqueous medium). The compositions can be sterile and can be in an injectable form (e.g., a form suitable for intravenous injection). The antibodies or fragments (or derivatives thereof) can also be formulated as a composition appropriate for topical administration to the skin or mucosa (e.g., intrarectal or intravaginal administration). Such compositions can take the form of liquids, ointments, creams, gels and pastes. The antibodies or fragments (or derivatives thereof) can also be formulated as a composition appropriate for intranasal administration. The antibodies or fragments (or derivatives thereof) can be formulated so as to be administered as a post-coital douche or with a condom. Standard formulation techniques can be used in preparing suitable compositions.

The bnAbs and fragments thereof (and derivatives) described herein have utility, for example, in settings including the following:

i) in the setting of anticipated known exposure to HIV-1 infection, the antibodies described herein, or fragments thereof, (or derivatives thereof) and be administered prophylactically (e.g., IV, topically or intranasally) as a microbiocide,

ii) in the setting of known or suspected exposure, such as occurs in the setting of rape victims, or commercial sex workers, or in any homosexual or heterosexual transmission without condom protection, the antibodies described herein or fragments thereof (or derivatives thereof) can be administered as post-exposure prophylaxis, e.g., IV or topically, and

iii) in the setting of Acute HIV infection (AHI), the antibodies described herein, or fragments thereof, (or derivatives thereof) can be administered, alone or in combination with another anti-HIV-1 therapeutic, as a treatment for AHI to control the initial viral load or for the elimination of virus-infected CD4 T cells.

Suitable dose ranges can depend on the antibody or fragment (or derivative thereof—e.g., toxin- or radioisotope-bound derivative) and on the nature of the formulation and route of administration. Optimum doses can be determined by one skilled in the art without undue experimentation. Doses of antibodies in the range of 1-50 mg/kg can be used. If, for example, antibodies or fragments, with or without toxins, are used or antibodies are used that can be targeted to specific CD4 infected T cells, then less antibody or fragment can be used (e.g., from 5 mg/kg to 0.01 mg/kg).

In accordance with the invention, the bnAbs or antibody fragments (or derivatives) described herein can be administered prior to contact of the subject or the subject's immune system/cells with HIV-1 or, for example, within about 48 hours of such contact. Administration within this time frame can maximize inhibition of infection of vulnerable cells of the subject with HIV-1.

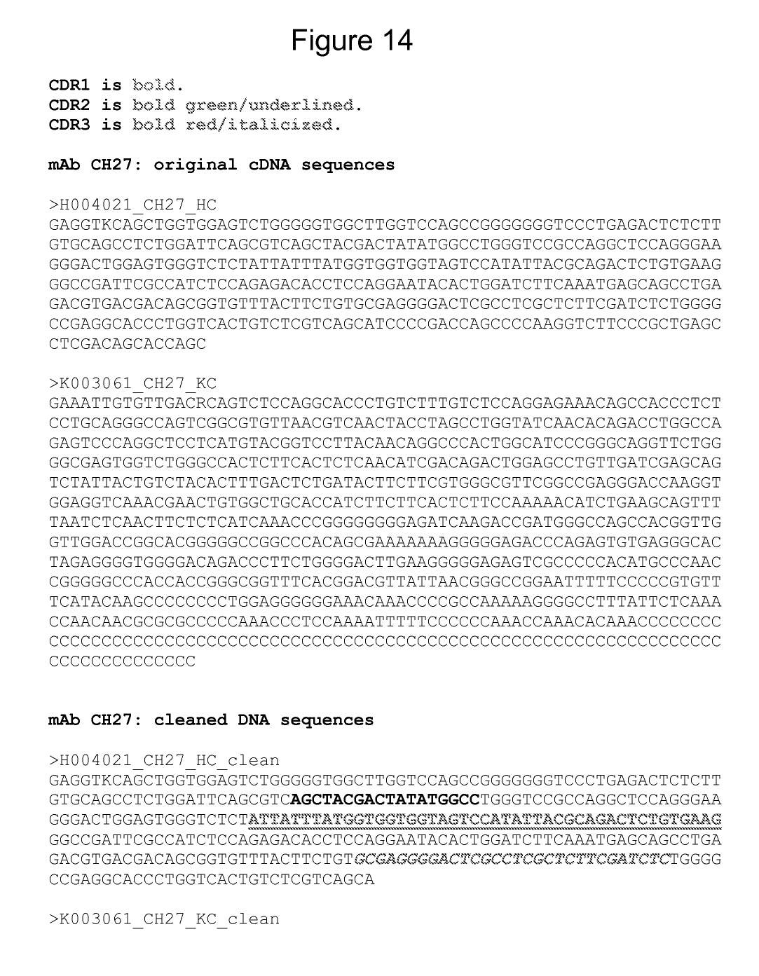

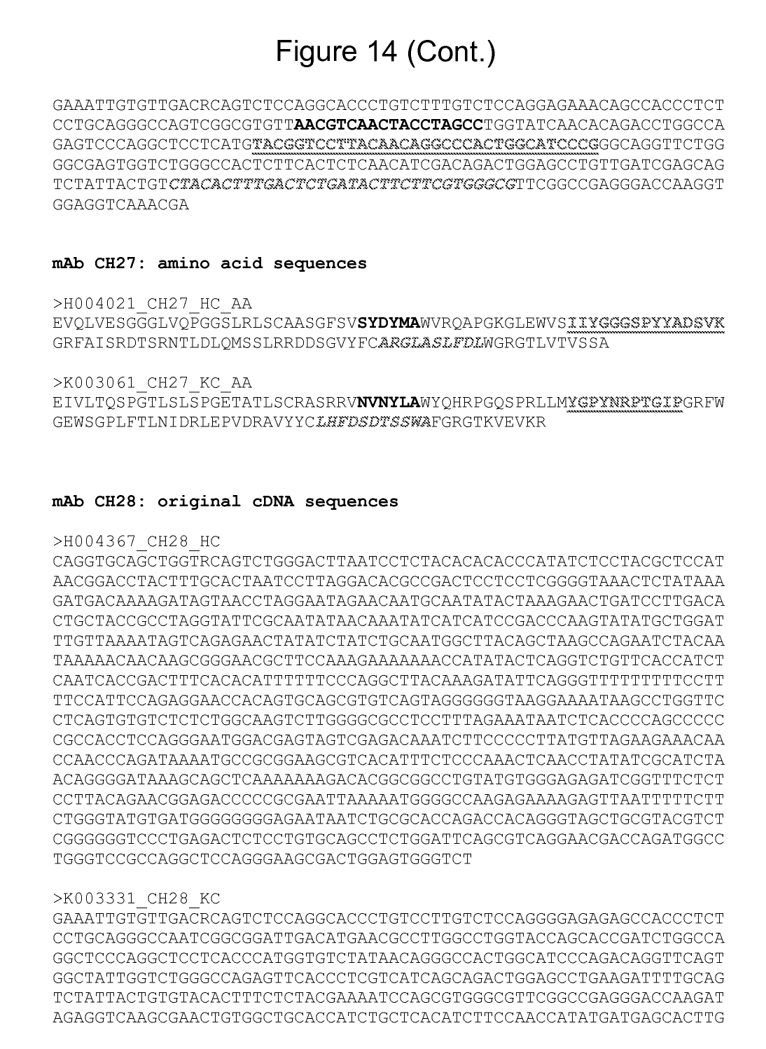

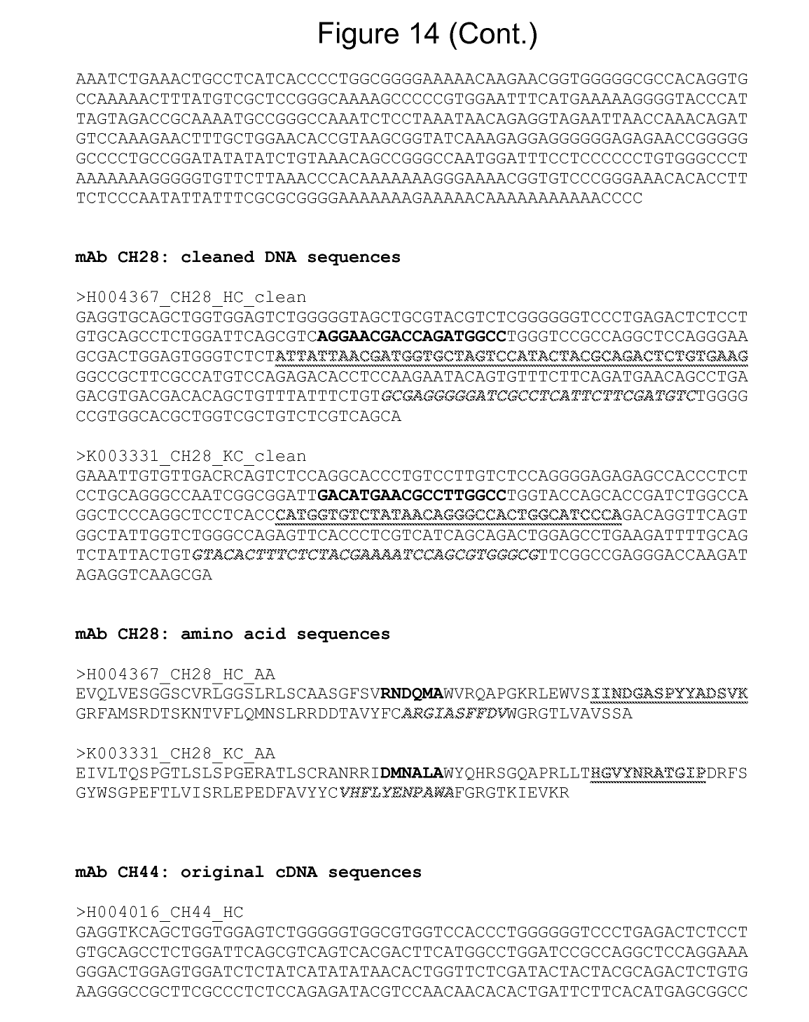

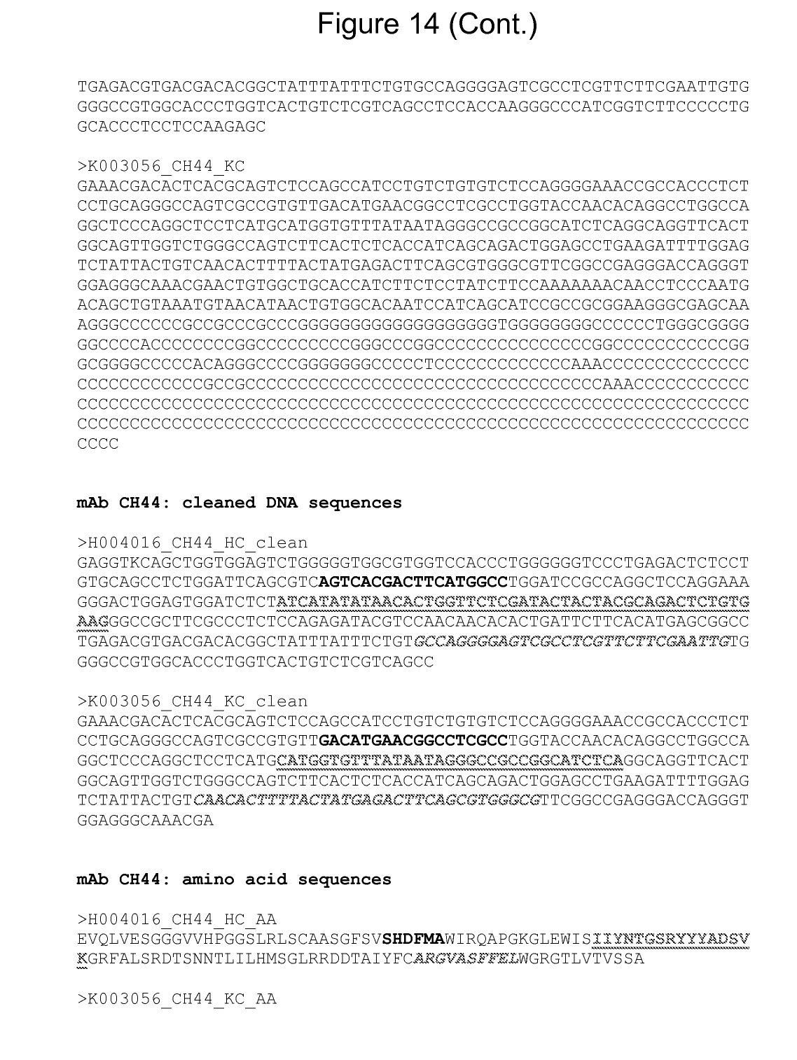

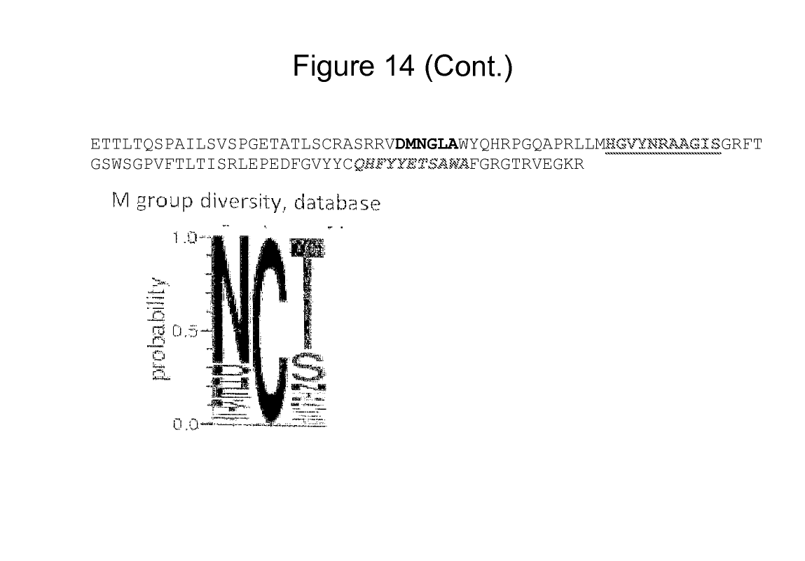

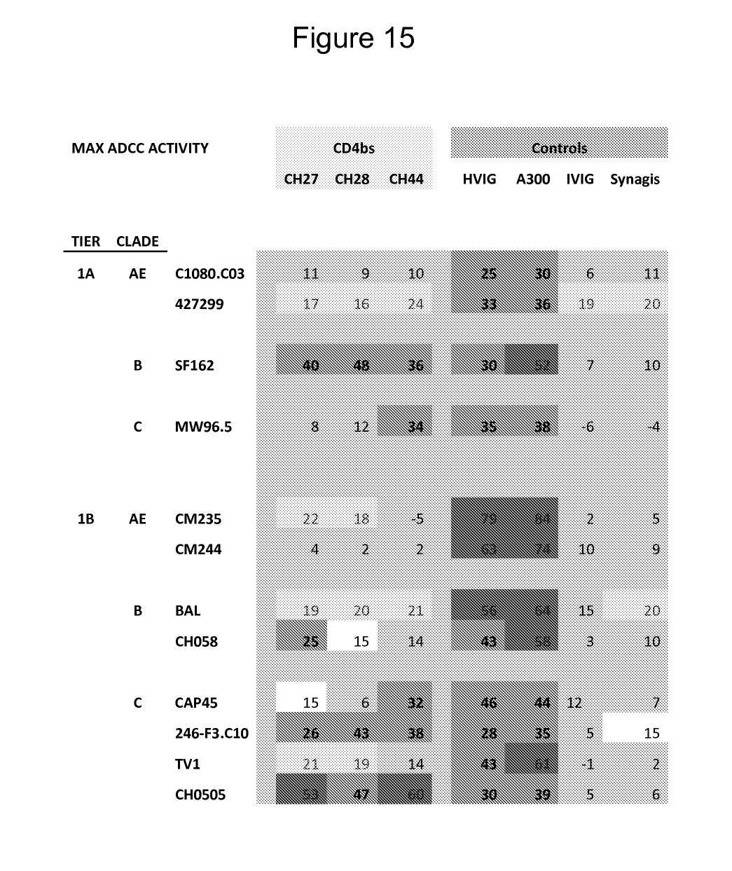

Antibodies of the invention and fragments thereof can be produced recombinantly using nucleic acids comprising nucleotide sequences encoding, for example, VH and VL chains (or CDRs) selected from those shown in FIG. 14.

The antibodies of the present invention can be used as probes to identify their specificity and to identify candidate immunogens that can elicit this new class of antibodies. Candidate immunogens can be selected based on binding to the antibodies and their inferred intermediates. Binding can be assessed, for example, using surface plasmon resonance, ELISA, and multiplex binding (Luminex-based) assays. In addition, binding activity can be assessed by testing for the ability of these antibodies to block the binding of other molecules, such as other antibodies, soluble CD4, or other molecules. Binding can also be assessed using functional assays such as neutraliztion or ADCC. The invention includes methods of identifying such immunogens and immunogens so identified.

Certain aspects of the invention can be described in greater detail in the non-limiting Example that follows. (See also Moody et al, Retrovirology 9 (Suppl 2): 035 (2012).)

In certain embodiments the invention provides antibodies with dual targeting specificity. In certain aspects the invention provides bi-specific molecules that are capable of localizing an immune effector cell to an HIV-1 envelope expressing cell, so as facilitate the killing of the HIV-1 envelope expressing cell. In this regard, bispecific antibodies bind with one “arm” to a surface antigen on target cells, e.g. HIV-1 envelope, and with the second “arm” to an activating, invariant component of the T cell receptor (TCR) complex, eg. CD3. The simultaneous binding of such an antibody to both of its targets will force a temporary interaction between target cell and T cell, causing activation of any cytotoxic T cell and subsequent lysis of the target cell. Hence, the immune response is re-directed to the target cells and is independent of peptide antigen presentation by the target cell or the specificity of the T cell as would be relevant for normal MHC-restricted activation of CTLs. In this context it is crucial that CTLs are only activated when a target cell is presenting the bispecific antibody to them, i.e. the immunological synapse is mimicked. Particularly desirable are bispecific antibodies that do not require lymphocyte preconditioning or co-stimulation in order to elicit efficient lysis of target cells.

In certain embodiments, such bispecific molecules comprise one portion which targets HIV-1 envelope and a second portion which binds a second target. In certain embodiments, the first portion comprises VH and VL sequences, or CDRs from CH27, 28, or CH44 (FIG. 14).

In certain aspects the invention provides use of the antibodies of the invention, including bispecific antibodies, in methods of treating and preventing HIV-1 infection in an individual, comprising administering to said individual a therapeutically effective amount of a composition comprising the antibodies of the invention in a pharmaceutically acceptable form. In certain embodiment, the methods include a composition which includes more than one HIV-1 targeting antibody. In certain embodiments, the HIV-1 targeting antibodies in such combination bind different epitopes on the HIV-1 envelope. In certain embodiments, such combinations of bispecific antibodies targeting more than one HIV-1 epitope provide increased killing of HIV-1 infected cells. In other embodiments, such combinations of bispecific antibodies targeting more than one HIV-1 epitope provide increased breadth in recognition of different HIV-1 subtypes.

### EXAMPLES

### Example 1

### HIV Neutralizing Antibodies without Heterologous Breadth can Potently Neutralize Autologous Viruses

Broadly neutralizing antibodies (bnAbs) against HIV-1 have activity in vitro against difficult-to-neutralize (tier 2) viruses while antibodies that arise following vaccination or early in HIV-1 infection have activity only against easy-to-neutralize (tier 1) viruses. The capacity for antibodies that neutralize only heterologous tier 1 viruses to exert selection pressure on HIV-1 is not known. To study this question, we isolated tier 1 virus-nAbs that bind to the third variable loop (V3) or the CD4 binding site (CD4bs) from two HIV-1-infected individuals and determined the antibody sensitivity of autologous HIV-1 strains sampled over time. We found functional autologous viruses could be neutralized by these V3 and CD4bs antibodies, and found that resistant forms of HIV-1 accumulated over time, suggesting Ab-mediated viral selection pressure. One clinical setting where transfer of both autologous nAbs and virus can occur is that of mother-to-child transmission (MTCT). In this setting, high levels of maternal V3 and CD4bs autologous nAbs may may be able to reduce transmission, regardless of autologous nAb breadth and potency against heterologous viruses.

Induction of antibodies with neutralization breadth is a primary goal of HIV-1 vaccine development (1). All current HIV-1 envelope (Env) immunogens frequently induce neutralizing antibodies (nAbs) that inhibit only easy-to-neutralize (tier 1) HIV-1 strains (2). In contrast, broadly neutralizing antibodies (bnAbs) that can potently neutralize a variety of difficult-to-neutralize (tier 2) HIV-1 strains that have been associated with HIV-1 transmission (3) are not induced by current vaccines (1, 2, 4, 5).

The initial autologous nAb response in HIV-1-infected subjects is generally restricted to neutralizing the infecting transmitted/founder virus (6-13). Epitopes frequently targeted by the initial autologous nAbs are the third constant region-variable loop 4 (C3-V4) domain (8, 10, 13), the base of the third variable (V3) loop (11, 12, 14), the first and second variable loop (V1V2) regions (9, 10, 12, 15), and the CD4 binding site (CD4bs) (16, 17). In chronic HIV-1 infection, virus escape mutants are selected that repopulate the plasma virus pool, and neutralization breadth accrues to varying degrees in different individuals (18). In addition, antibodies to V3 and the CD4bs arise that can neutralize heterologous tier 1 but not tier 2 HIV-1 isolates (2, 19-24). However, the neutralization sensitivity of the autologous repopulated plasma virus pool to this type of V3 and CD4bs nAbs has not been studied. Here, we have isolated from two chronically HIV-1-infected individuals V3 and CD4bs nAbs with breadth only for tier 1 but not tier 2 heterologous viruses, and for comparison, CD4bs bnAbs with tier 2 neutralization breadth; and determined the ability of these Abs to neutralize a large pane of autologous viruses as well as to select virus escape mutants.

Isolation of nAbs with Restricted or Broad Neutralizing Activity from Chronically Infected Individuals.

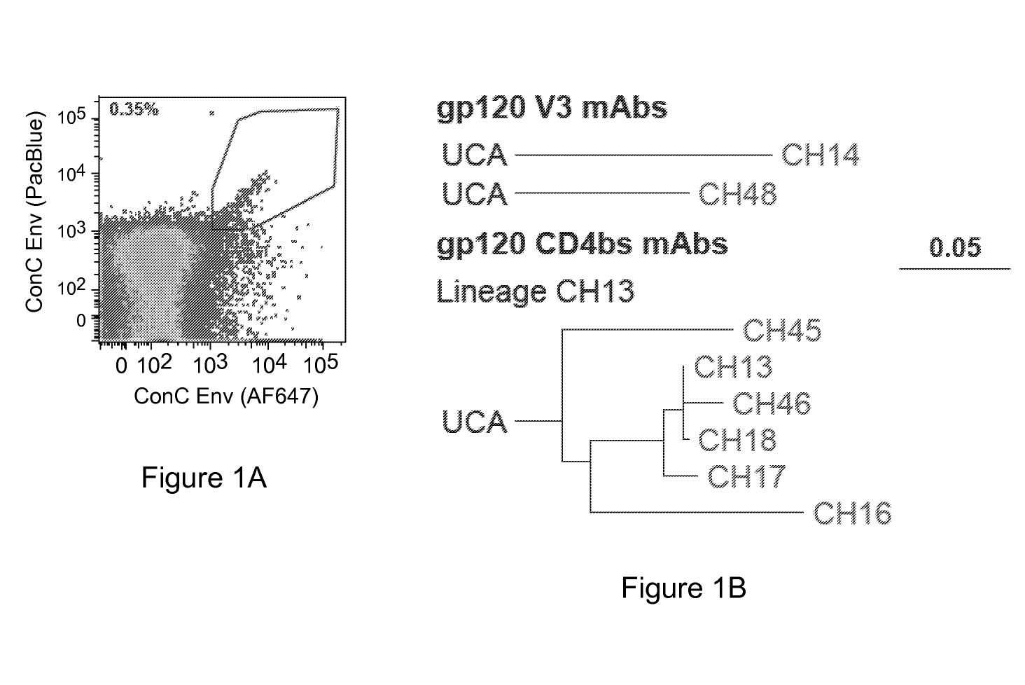

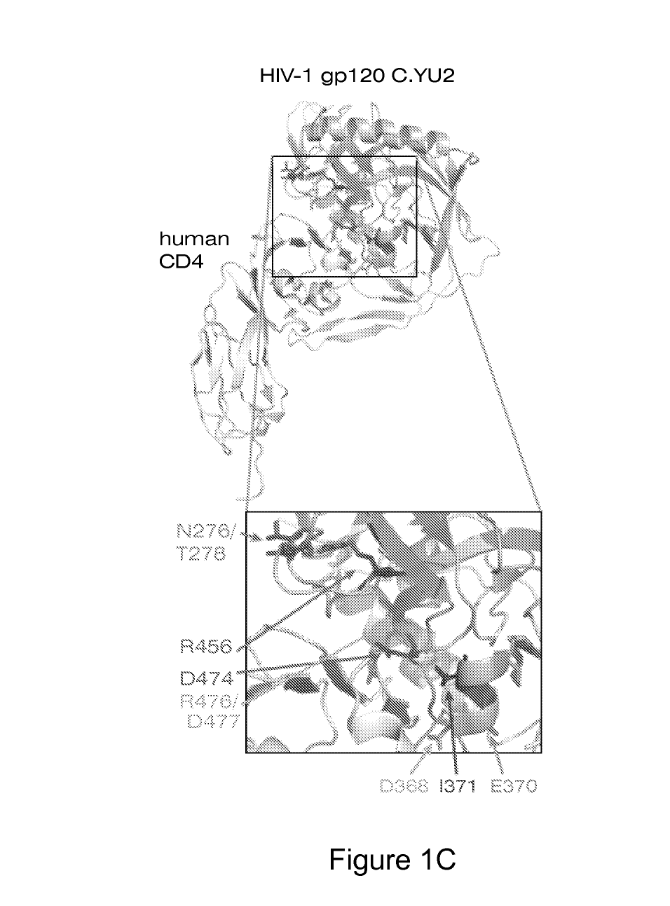

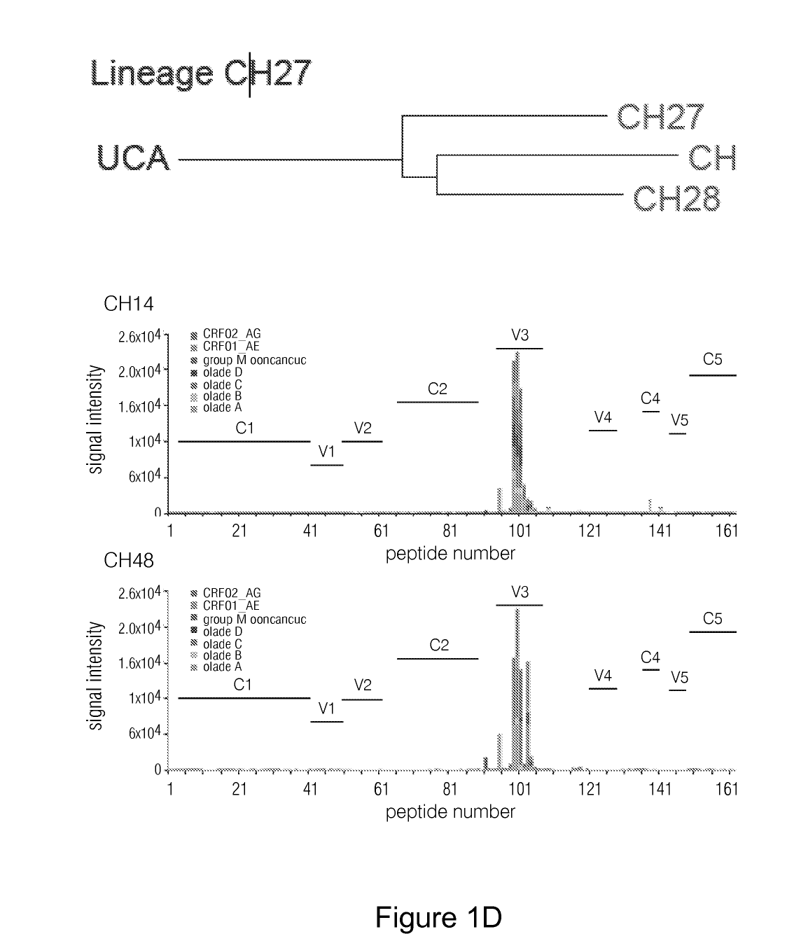

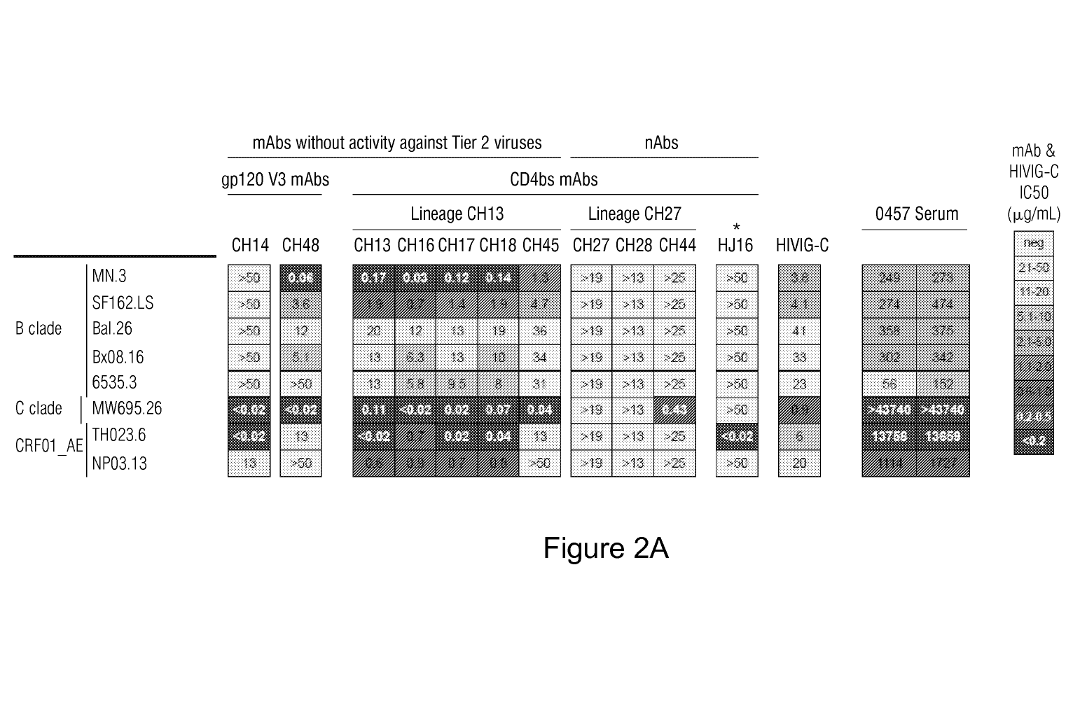

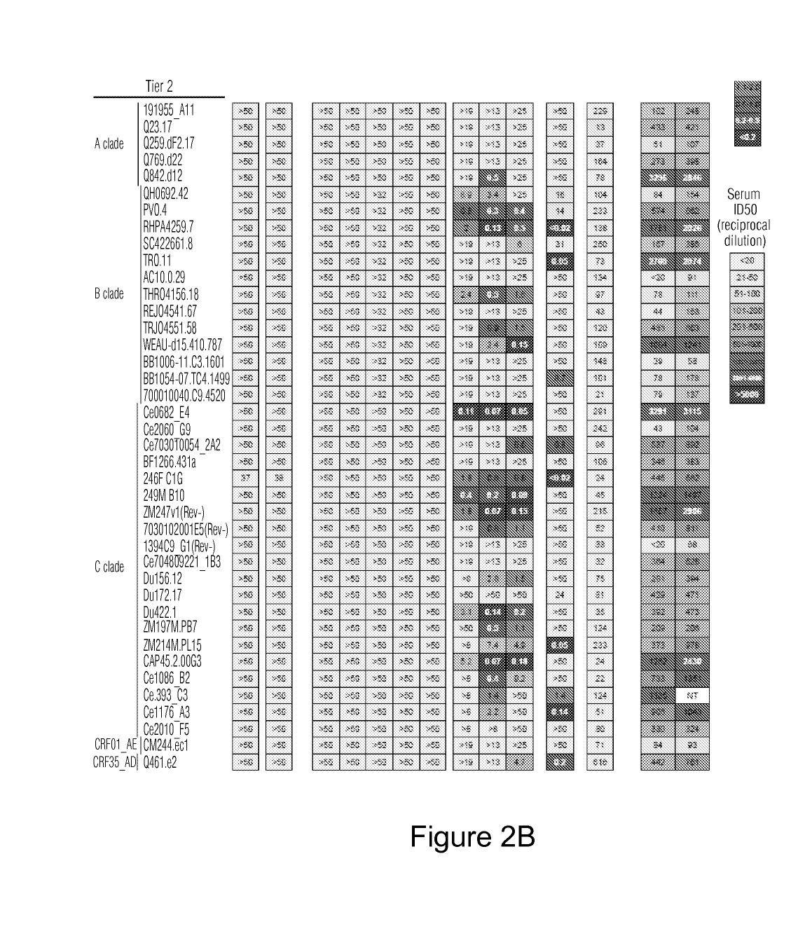

From chronically HIV-1 infected individual CH0457, we isolated two clonal lineages as well as single monoclonal antibodies (mAbs) using antigen-specific memory B cell flow cytometry sorting (FIG. 1a, 1b; Table S1). Epitope mapping with virus mutants demonstrated that the CH13 lineage mAbs (CH13, CH16, CH17, CH18, CH45) bound to the CD4bs (FIG. 1c; Tables S2 and S3), and neutralization assays demonstrated that members of the lineage neutralized 8/8 tier 1 heterologous HIV-1 Env pseudoviruses, but did not neutralize any of 26-40 tier 2 heterologous HIV-1 Env pseudoviruses (FIG. 2). Two additional mAbs, CH14 and CH48, were not clonally related, and both mAbs mapped to the HIV-1 Env V3 loop (FIG. 1d; Table S4). Like the CD4bs clonal lineage CH13, V3 mAbs CH14 and CH48 neutralized tier 1 but not tier 2 heterologous HIV-1 strains (FIG. 2).

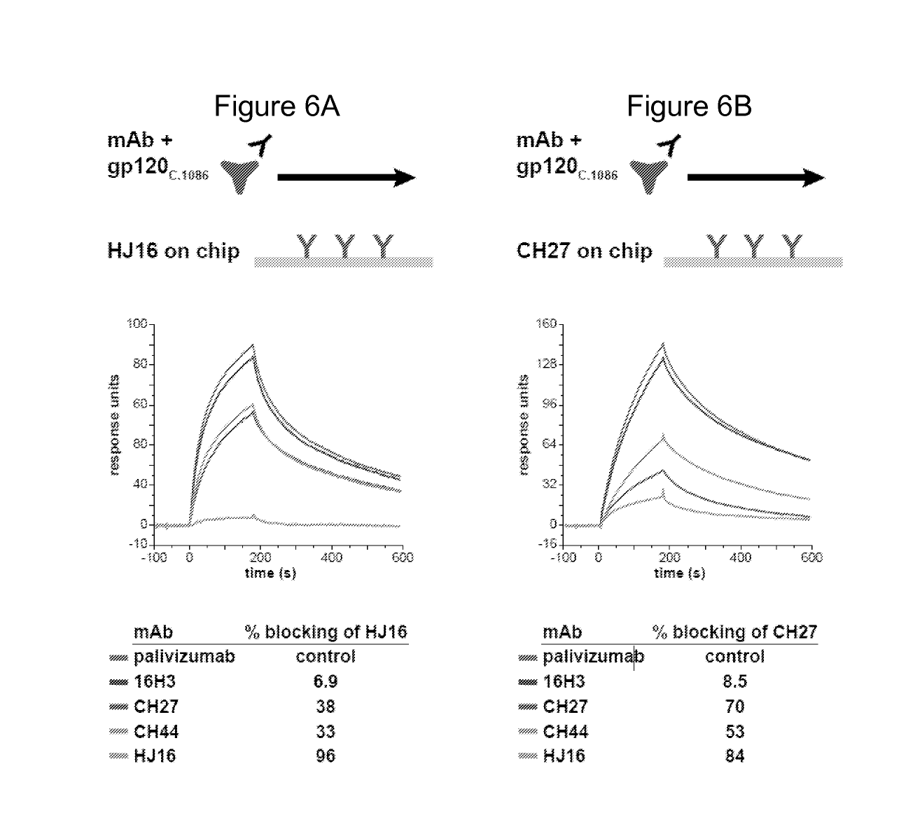

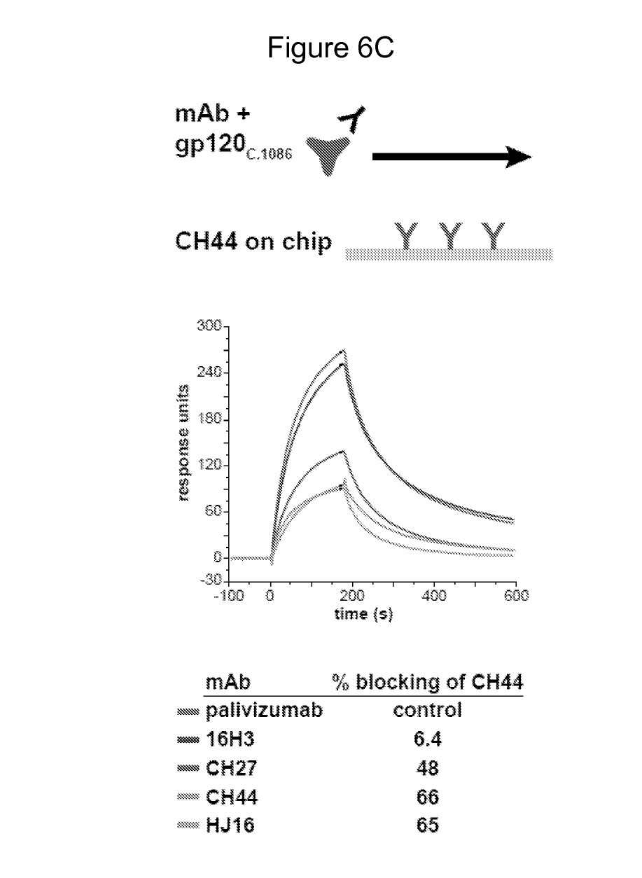

The second clonal lineage of mAbs from CH0457, CH27 (FIG. 1b), had two members (CH27 and CH28) that were IgA2 while the third (CH44) was IgG1 (Table S1). Neutralization assays with clonal lineage CH27 mAbs showed that all three lineage members (CH27, CH28, CH44) neutralized 40% (range 25-48%) of 40 tier 2 heterologous HIV-1 strains (FIG. 2). The CH27 lineage mAbs preferentially neutralized tier 2 but not tier 1 heterologous viruses. HJ16 is a CD4bs bnAb isolated from another infected individual (25) and like the CH27 lineage mAbs, HJ16 neutralizes multiple tier 2 but not tier 1 viruses. Mutation of Env at N276 conferred resistance to HJ16 (26), and mAbs of the CH27 lineage were similarly sensitive to mutations at N276 and T278 (Table S5). CH27, CH44, and CD4bs nAb HJ16 (26) cross-blocked each other in Env binding assays (FIG. 6), demonstrating that the CH27 lineage antibodies were similar to HJ16 (FIG. 2). Serum from chronically-infected individual CH0457 taken from weeks 8 and 96 of observation were tested against the same panel of heterologous viruses (FIG. 2). Neutralization titers and breadth against heterologous viruses were very similar at the two chronic infection time points (R²=0.95, Pearson's correlation p<2.2×10⁻¹⁶).

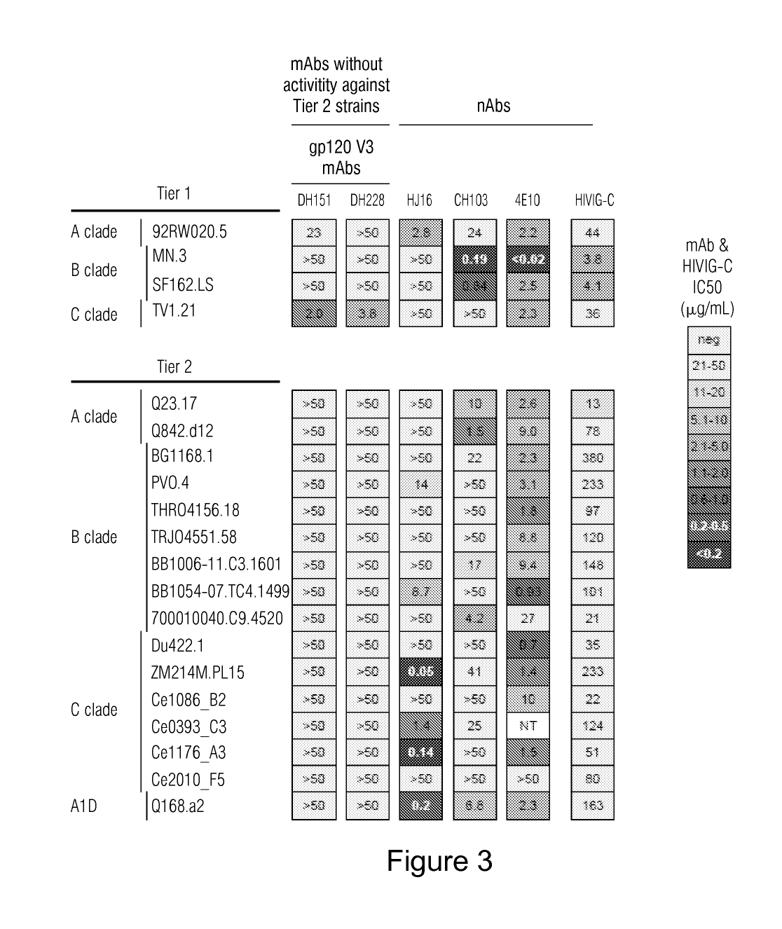

From a second individual, CH505, previously described to have a CD4bs bnAb lineage (represented by CH103 in FIG. 3) (16), we isolated two V3 nAbs (DH151 and DH228; Table S6) from 41 weeks after transmission (FIG. 3). The neutralization patterns exhibited by nAbs DH151 and DH228 were similarly restricted to a subset of tier 1 heterologous viruses, and they did not neutralize any of 16 tier 2 heterologous viruses (FIG. 3).

Virus Evolution in Chronically Infected Individual CH0457.

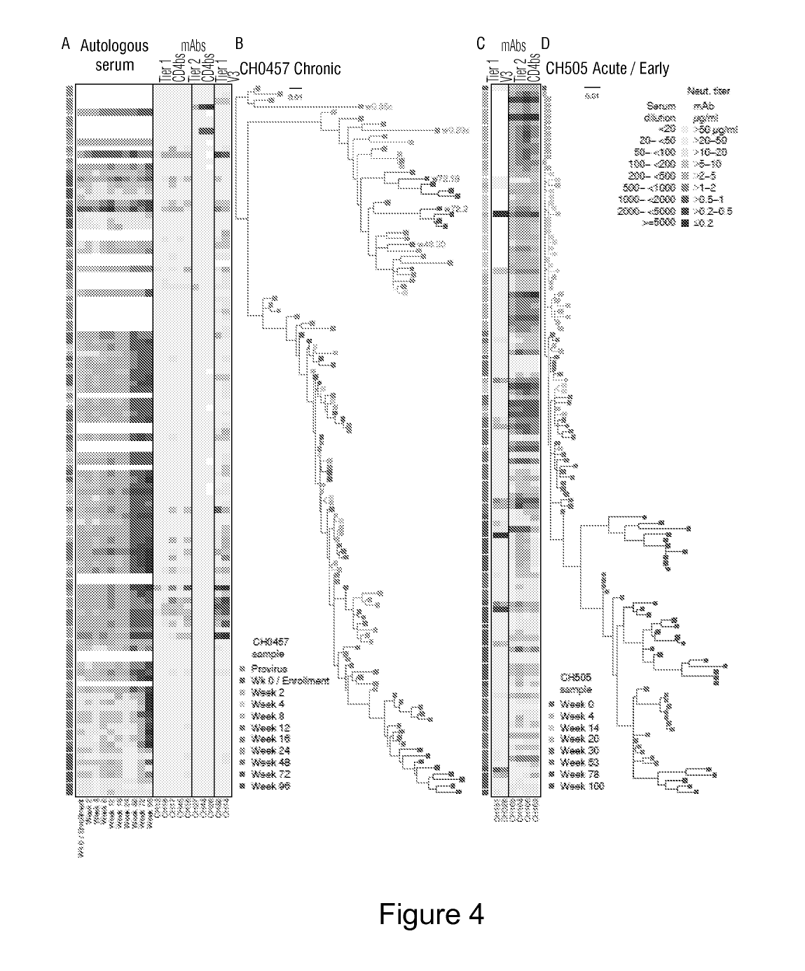

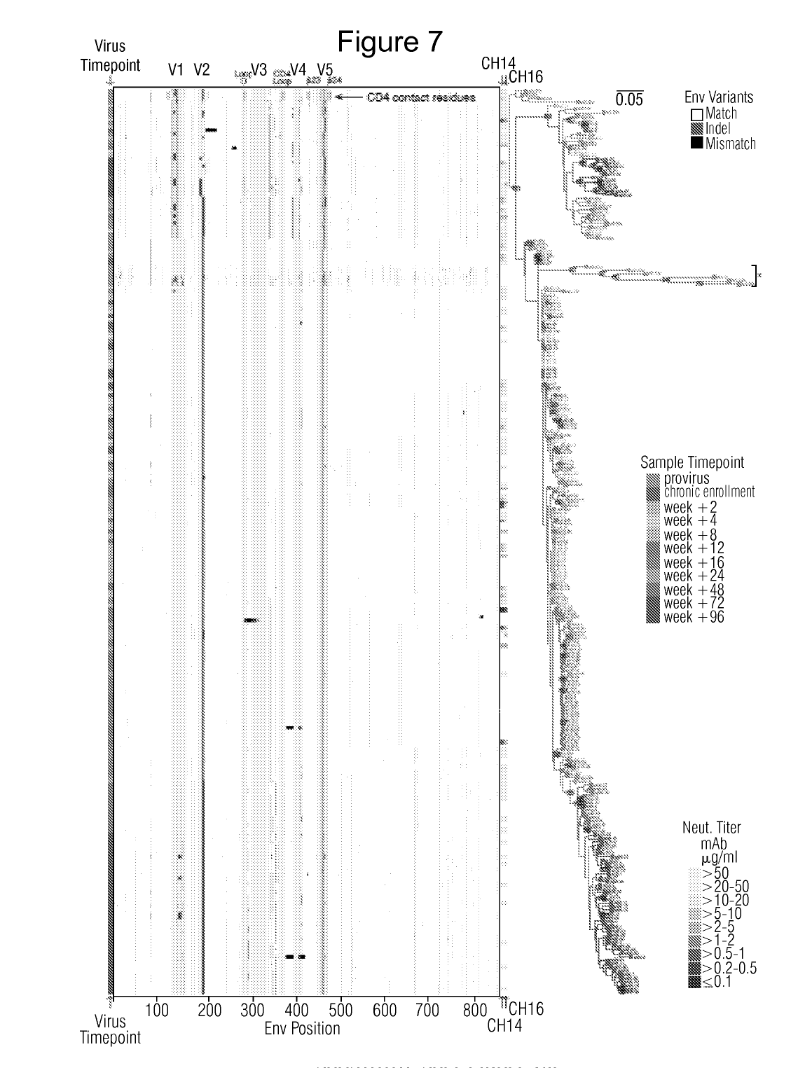

We amplified a total of 209 CH0457 env gene sequences by single genome amplification (SGA) from 10 time points over a two year period during chronic infection (weeks 0, 2, 4, 8, 12, 16, 24, 48, 72, and 96 post-enrollment). An average of 21 (range 12-35) SGA env sequences were analyzed for each time point. Phylogenetic analysis showed that the Env sequences continuously evolved over time (FIG. 7). The Env sequences from weeks 48, 72 and 96 were more divergent compared with the earlier viruses (0 to week 16) (FIG. 7). Furthermore, within-subject phylogeny maintained a persistent minority clade that represented a small fraction (average 14%) of Envs sampled at any given time point (FIG. 4; FIG. 7) throughout the study period. The consensus of this clade differed at 85/888 (9.6%) aligned Env amino acid positions from the consensus of the main clade. Phylogenetic analysis and BLAST searching of sequences from CH0457 relative to the database indicated that despite the genetic distance, the sequences from this minor persistent clade were more closely related other sequences from CH0457 than to other strains, and validated that this clade was not a contamination event, nor was it evidence of super-infection with two distinct viruses. Rather the major and minor clades emerged from a common founder in CH0457.

Neutralization of Autologous Viruses by bnAbs and Tier 1 Virus-Neutralizing mAbs.

We made 84 pseudoviruses from these env sequences (FIG. 4B; average 8 per time point; range 7-11) for neutralization assays against CH0457 serum samples (FIG. 4A). The serum from later time points (weeks 72 and 96) potently neutralized the early viruses (week 48 or earlier) but not the later viruses, indicating that autologous nAbs were continuously elicited during chronic infection in CH0457 (FIGS. 4A and 4B).

We next determined the neutralization activities of the CH27 CD4bs lineage bnAbs against the panel of 84 autologous pseudoviruses derived from viral RNA from plasma samples. Five autologous viruses were weakly neutralized by one of three lineage CH27 bnAbs (range 32-50 μg/mL), while the other 79 pseudoviruses (94%) were resistant to the CH27 lineage bnAbs (FIG. 4A; FIG. 8A). These data suggested that the autologous virus population in this individual by the time of enrollment had already escaped from pressure exerted by the CH27 lineage of bnAbs, with viral escape occurring during chronic infection prior to study enrollment.

Thus, to seek definitive evidence of evolutionary selection exerted by the CH27 bnAb lineage, we amplified proviral env genes archived in peripheral blood mononuclear cells (PBMC) from the earliest time point (termed week 0) in this study. Like plasma-derived Env pseudoviruses, the majority of the PBMC-derived Env pseudoviruses were resistant to the lineage CH27 bnAbs (FIGS. 4A and 4B). However, two cell-derived Env pseudoviruses (w0.35c and w0.29c) were found to be highly sensitive to the lineage CH27 bnAbs, thus documenting CH27 bnAb lineage-mediated escape (FIG. 4A; FIG. 8B). Remarkably, both of these viruses sensitive to the CH27 lineage were members of the persistent minority clade (FIG. 4B; FIG. 7). Of note, the archived proviral DNA sequences recapitulated evolutionary intermediates reconstructed from the sequence data that represented transition forms between the two CH0457 viral clades.

Next, we asked if 7 of the CH0457 tier 1 virus-nAbs (5 CH13 lineage CD4bs mAbs, and 2 V3 mAbs CH14 and CH48) could neutralize autologous HIV-1 pseudoviruses. We found that the V3 and CD4bs mAbs were able to neutralize autologous viruses throughout the 2-year study period, including PBMC-archived viruses (FIGS. 4A and 4B; FIG. 8). Remarkably, the tier 1 virus-neutralizing CD4bs clonal lineage CH13 mAbs neutralized 52/84 (62%) autologous plasma viruses and 11/34 (32%) of autologous PBMC viruses, while the V3 tier 1 virus-neutralizing mAbs (CH14 and CH48) neutralized 67/84 (80%) autologous plasma viruses and 28/34 (82%) of autologous PBMC viruses. Neutralization potency ranged from 50 μg/mL to 0.06 μg/mL, with 21/257 (8%) neutralization assays of tier 1 virus-neutralizing antibodies demonstrating neutralization of autologous viruses at ≦2 μg/mL.

Sensitivity to the CH13 lineage and to the two V3 mAbs peaked at week 24 after enrollment; by week 48 of follow-up, most viruses were resistant to the V3 mAbs (FIG. 4A; FIG. 8A online) suggesting selection of escape mutants by these nAbs. Of note, among the viruses sampled between weeks 48 and 96, only three viruses were still moderately sensitive to these nAbs (w48.20, w72.2, and w72.18), with the rest only weakly sensitivity or completely resistant.

The 3 CH0457 viruses sensitive to the CD4bs CH13 lineage (w48.20, w72.2, and w72.18) were all located within the persistent minor clade (3/10 in the minor clade vs. 0/17 in the dominant clade; Fisher's exact test p=0.04). The fact that both in the CDbs CH27 lineage and in the CH13 lineage the sensitive viruses persisted longest in the minor clade but not in the dominant clade raises the possibility that the viruses in the minor clade may be emerging from an immunologically protected site (eg, brain or the CD4 T cell latent pool) where antibody pressure would be limited (27). Across all time points, 32/84 (38%) of autologous pseudoviruses were resistant to the CD4bs nAbs while 17/84 (20%) were resistant to the V3 loop mAbs. Analysis of CH0457 Env sequences did not demonstrate an accumulation of Env mutations at the putative nAb contact sites suggested by epitope mapping (Tables S2, S3, S4).

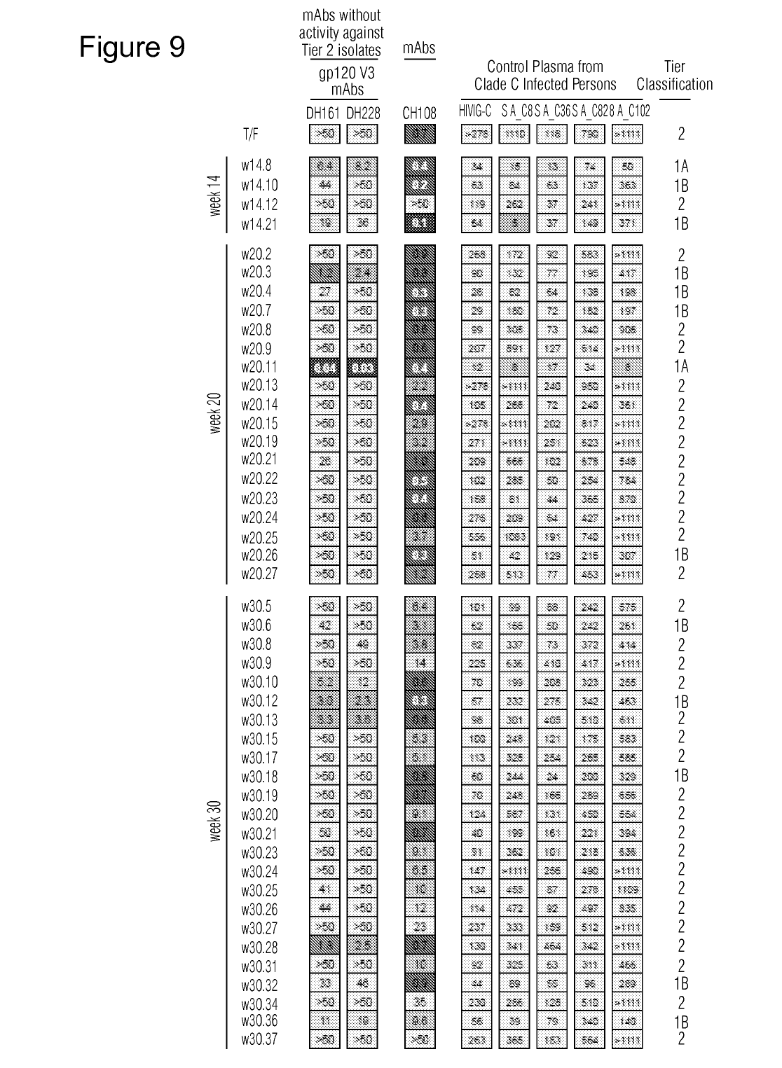

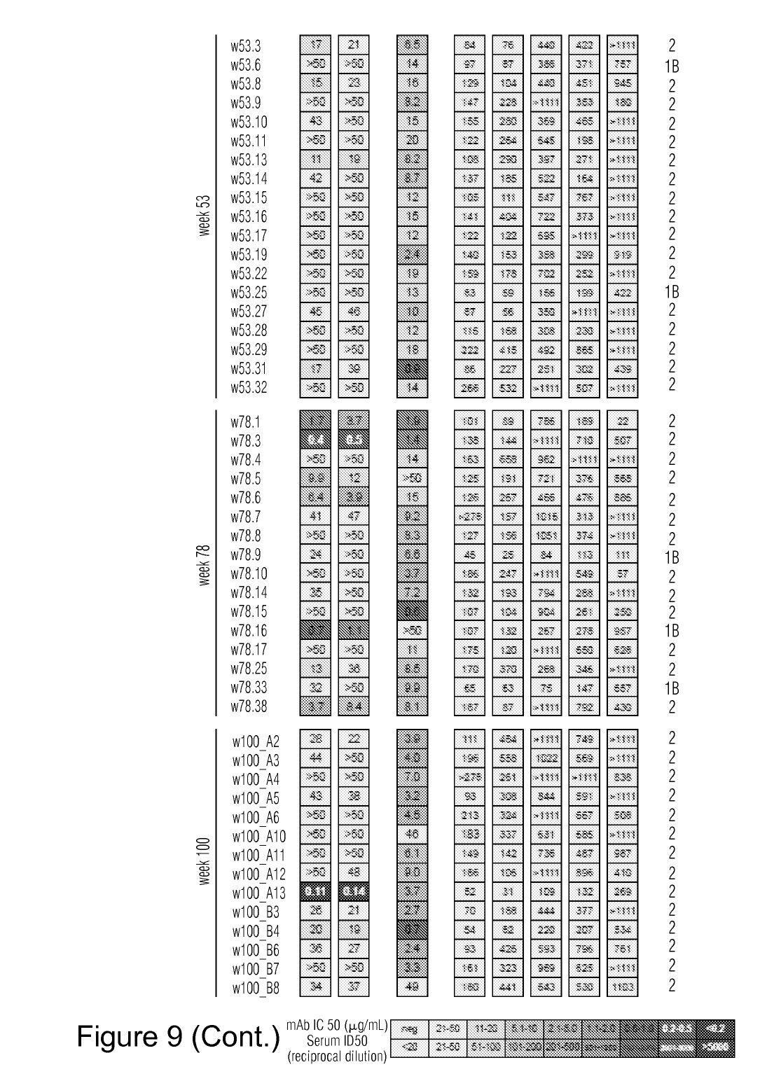

To determine if autologous virus neutralization by autologous tier-1 virus nAbs was a phenomenon unique to individual CH0457, we studied two V3 nAbs (DH151 and DH228; Table S6) isolated from a second HIV-1-infected African individual, CH505, 41 weeks after transmission (16). CH505 also developed a CD4bs clonal lineage (termed CH103) at 136 weeks after transmission (16). CH505 was studied earlier during infection compared with CH0457, thus Env selection by bnAbs was ongoing in individual CH505 at the time of study and many autologous Env pseudoviruses were only partially resistant to the CH103 bnAb lineage (Fig. S4) (28). Whereas both CH505 V3 mAbs neutralized a subset of tier 1 heterologous viruses, they did not neutralize any of 16 tier 2 heterologous viruses (FIG. 3). However, V3 mAbs DH151 and DH228 neutralized 45/96 (47%, IC₅₀ range 50-0.03 lμg/mL) autologous CH505 viruses (FIG. 4C; FIG. 9), and potently neutralized 7/96 (7.3%) viruses at <2 μg/mL. Interestingly, the transmitted/founder virus from CH505 was resistant to both V3 nAbs but became sensitive by week 14 after infection (FIGS. 4C and 4D; FIG. 9), suggesting that an escape mutant of the transmitted/founder elicited these V3 nAbs. Moreover, these data demonstrated viral Env V3 loop epitope exposure by week 14 after infection. As with the CH0457 individual, CH505 viruses sensitive to the V3 mAbs were present throughout all time points studied. Thus, CH505 V3 mAbs DH151 and DH228 had no neutralizing activity against heterologous tier 2 viruses but were able to neutralize autologous CH505 viruses, indicating that this phenomenon was not limited to the chronically HIV-1-infected individual CH0457.

Autologous Virus Neutralization Sensitivity.

To assess the susceptibility of autologous viruses to heterologous nAbs, we performed neutralization assays with a panel of tier 1 virus-neutralizing antibodies and bnAbs. Of 84 CH0457 autologous pseudoviruses, 73 (87%) were sensitive to the heterologous VRC01-like CD4bs bnAb CH31 (29) (FIG. 8A). Similarly 55/62 (89%) of viruses were sensitive to the loop binding CD4bs bnAb CH106 (16) (FIG. 8A). Glycan-dependent bnAb HJ16 (25) neutralized only 5/72 (7%) of viruses, consistent with escape of these autologous viruses from the clonal lineage CH27 nAbs (FIG. 6, Table S5).

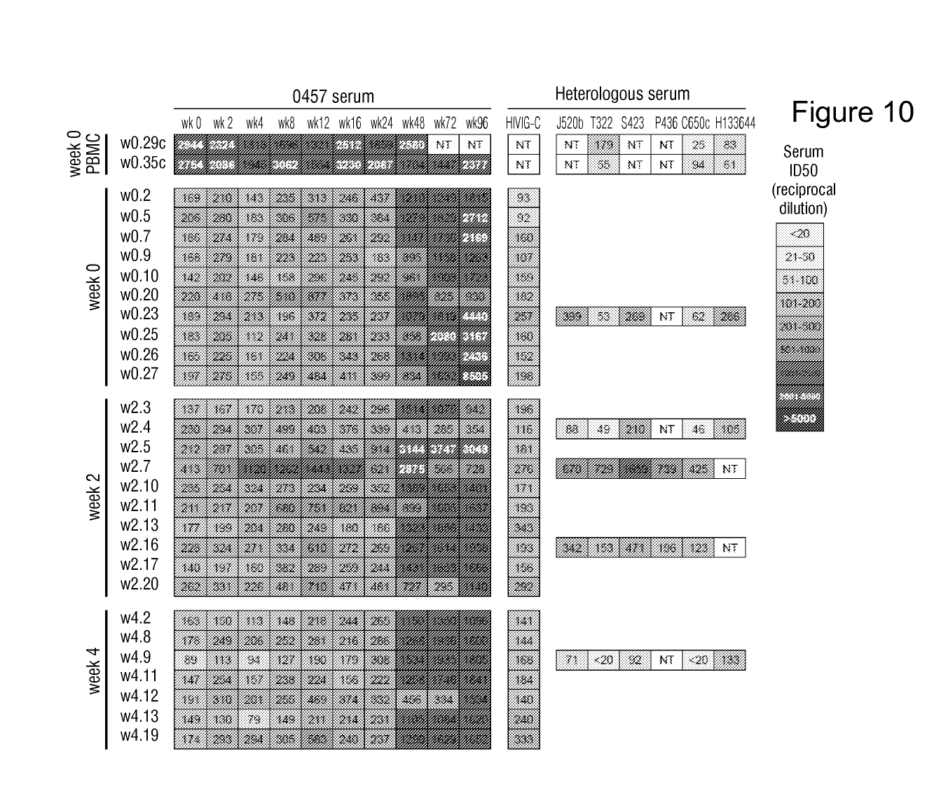

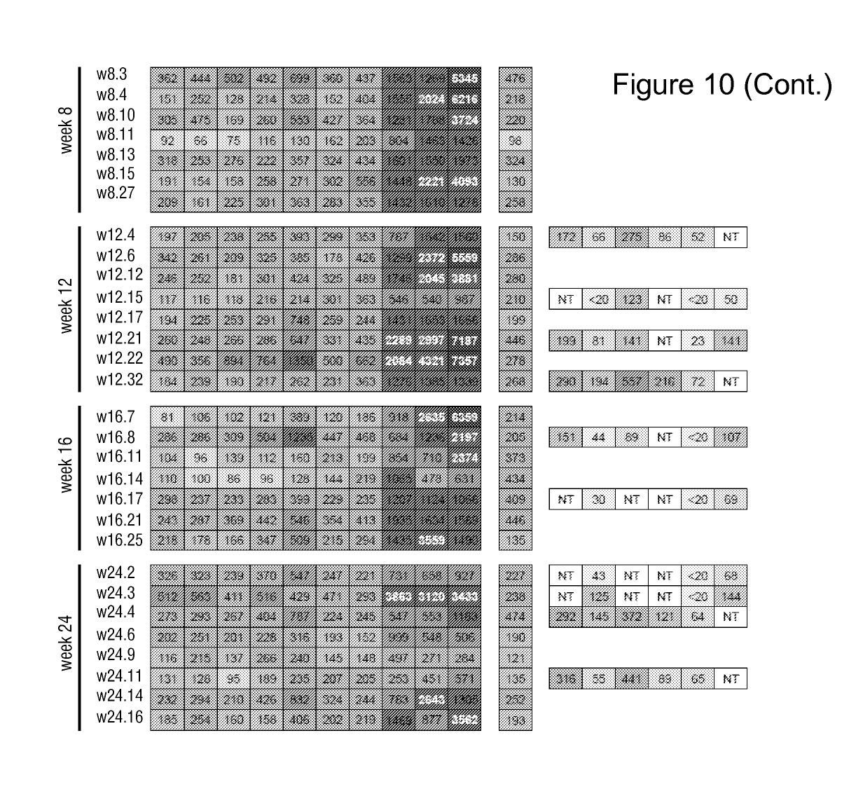

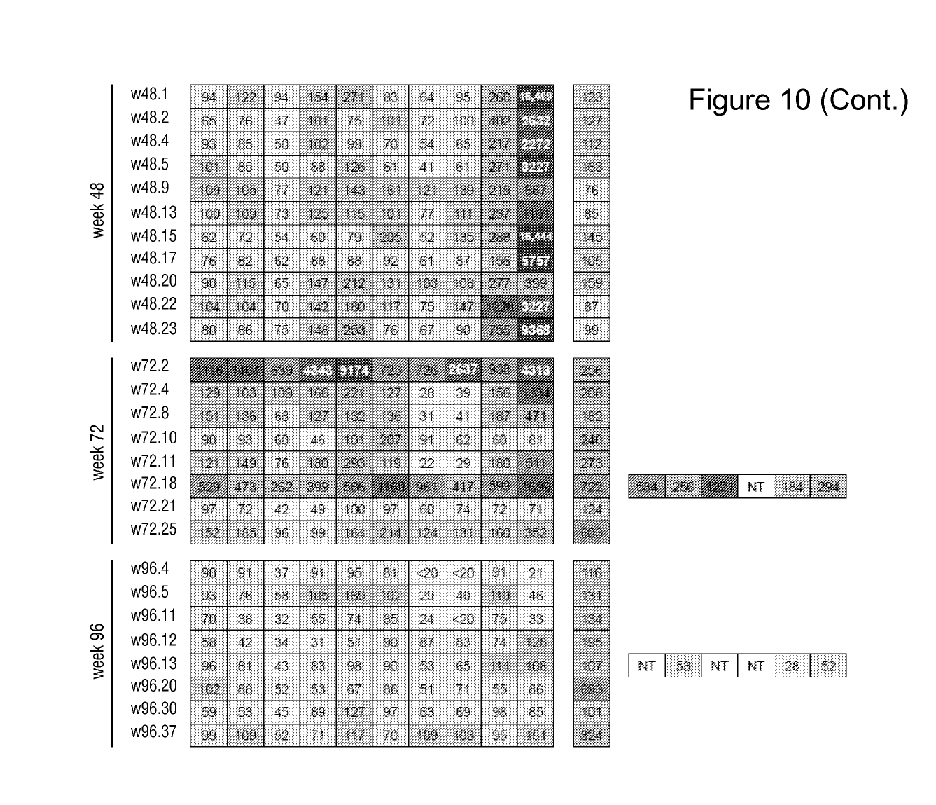

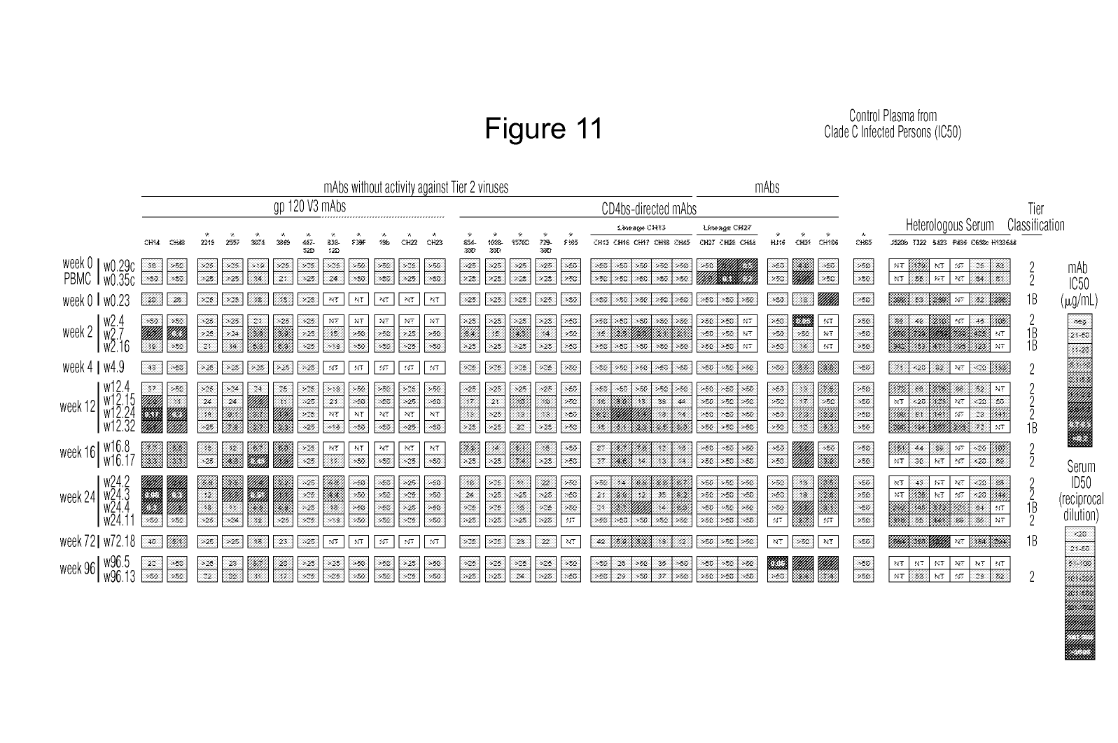

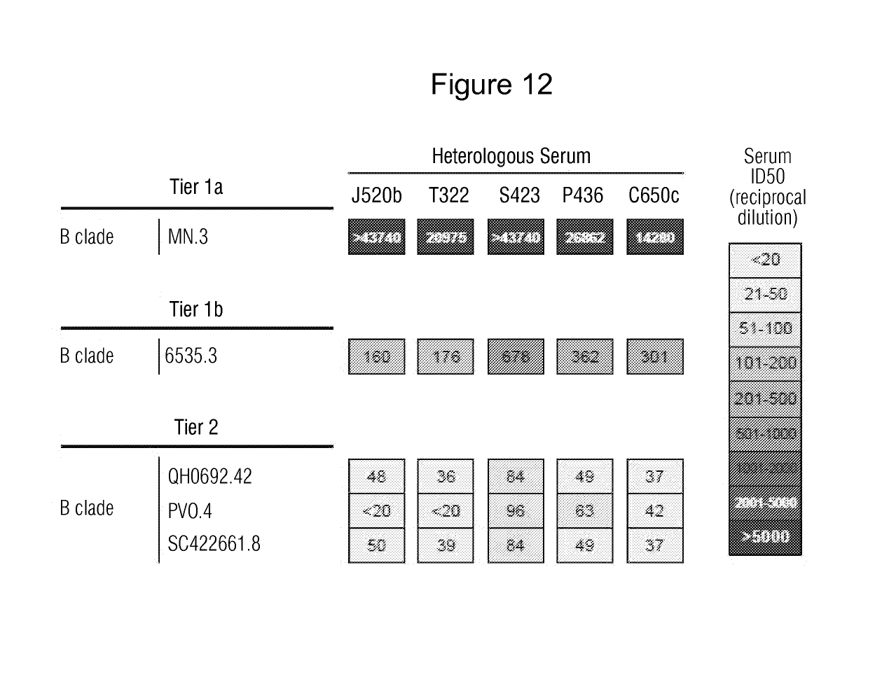

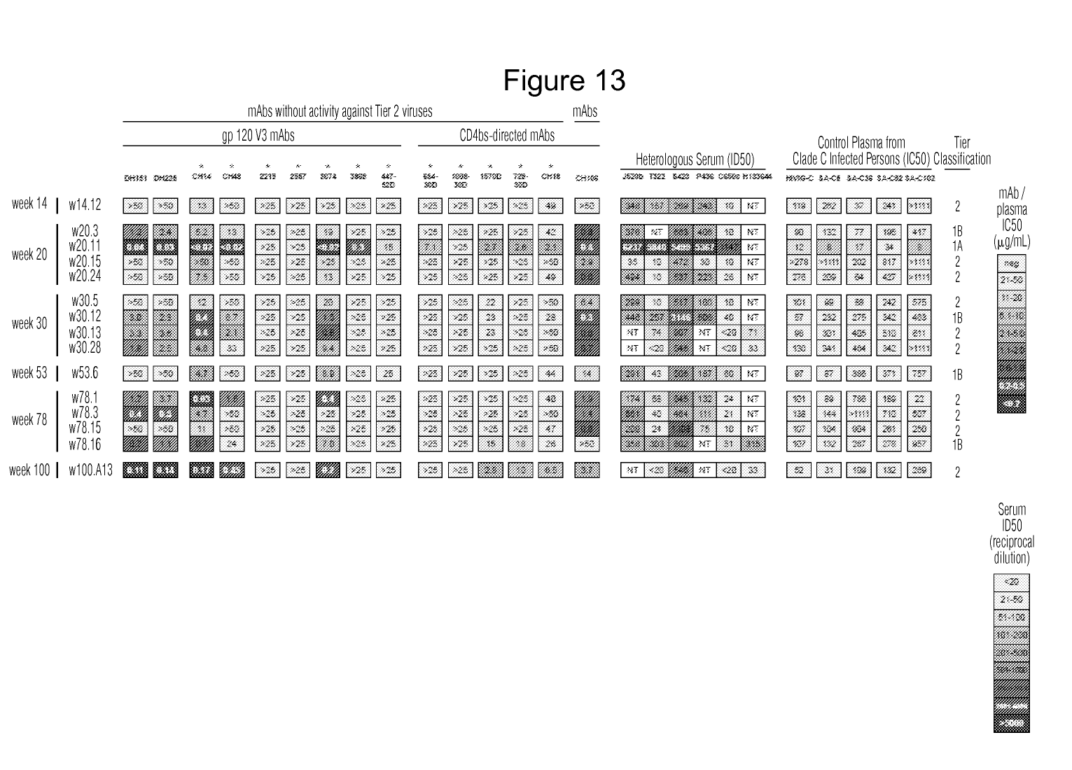

Next, we tested each of the 84 CH0457 Env pseudoviruses against the pooled serum product HIVIG-C and a subset of Env pseudoviruses against well-characterized HIV-1 patient serum samples (FIG. 10). The neutralization data suggested that CH0457 viruses sensitive to the autologous mAbs (CH13 lineage, CH14, and CH48) had exposed V3 and CD4bs epitopes. Thus, we analyzed a subset of Env pseudoviruses (10 sensitive and 10 resistant to autologous V3 and CD4bs nAbs) against a large panel of heterologous V3 and CD4bs mAbs previously shown to lack the ability to neutralize tier 2 virus isolates (2, 19-24) (FIG. 11). The 10 viruses sensitive to autologous nAbs were neutralized by this panel of heterologous V3 and CD4bs nAbs, suggesting that the V3 loop and CD4bs epitopes were indeed trimer-surface exposed. The 10 viruses resistant to autologous nAbs were also resistant to the heterologous nAb panel (FIG. 11). Testing of the same viruses using a panel of neutralization typing sera from HIV-1 infected persons showed that viruses with sensitivity to heterologous nAbs had an intermediate sensitivity to the typing sera (FIG. 11) consistent with an intermediate (tier 1B) (30) neutralization sensitivity phenotype (FIG. 12). Testing of autologous viruses from CH505 using a similar panel demonstrated predominant tier 1B neutralization sensitivity as well (FIG. 13). These data demonstrated that viruses arose in chronic infection in African individuals CH0457 and CH505 that could be neutralized by autologous V3 and CD4bs nAbs that themselves lacked tier 2 virus neutralization activity.

The initial autologous neutralizing antibody response that arises in acute HIV-1 infection is specific for the autologous virus with little tier 1 autologous virus breadth (31-33). This response differs from the autologous nAb response in chronic infection where breadth for heterologous tier 1 viruses can develop. When autologous neutralizing antibodies begin to show heterologous tier 1 breath, it is possible that such antibodies may be enroute to developing some degree of bnAb activity as occurred in the CH103 CD4bs lineage (16).

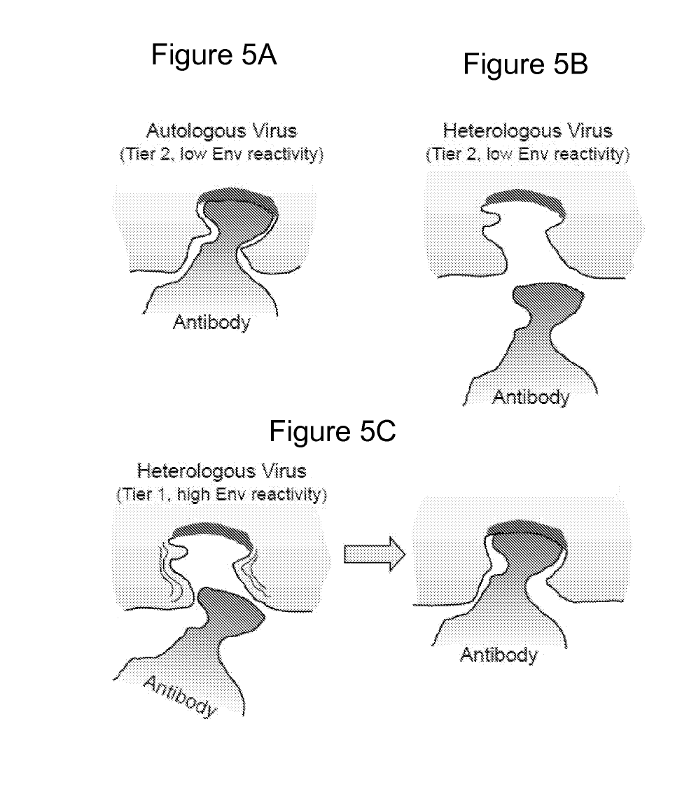

The CD4bs and V3 antibody lineages studied here were able to neutralize tier 1B and select tier 2 autologous HIV-1 isolates. We speculate that this was possible because the mAbs and viruses isolated in the present study co-evolved in the same HIV-1-infected individuals. During HIV-1 infection, virus quasispecies evolve that have different degrees of Env reactivity; viruses with high intrinsic activity (ie, tier 1A viruses) (30) are more reactive with both soluble CD4 and neutralizing antibodies (34). Thus, in these individuals, autologous viruses with low Env reactivity (ie, tier 1B or tier 2 viruses) (34, 35) can act as templates for antibody evolution, giving rise to antibodies that bind and neutralize autologous virus Envs with low reactivity (FIG. 5A). Such antibodies could broadly react with heterologous tier 1A Envs that have high reactivity (FIG. 5B), but would be expected to bind poorly to heterologous tier 2 Envs with low reactivity (FIG. 5C).

The ability of autologous neutralizing antibodies that arise in acute HIV-1 infection to exert immune pressure has been demonstrated by studies of the evolution of transmitted/founder viruses and plasma antibodies (31, 33, 36). In particular, the initial autologous-specific neutralizing antibody response to HIV-1 appears within the first year of infection and is associated with the development of resistant viruses in virtually all infected individuals (31, 33). A critical question is why neutralization of autologous viruses by tier 1 heterologous virus-neutralizing antibodies like the CH13 lineage from CH0457 and DH151 and DH228 V3 mAbs from CH505 has not been previously observed? The simplest answer is that testing of a large series of autologous Envs isolated in the setting of a chronically infected individual from whom multiple specificities of recombinantly-produced neutralizing mAbs have also been isolated has not been performed.

To date, HIV-1 vaccine efficacy trials have not convincingly demonstrated a protective effect of vaccine-elicited tier 1 virus-neutralizing antibodies (37, 38). In particular, the only vaccine study to date that demonstrated a degree of protection, the RV144 trial, did not elicit bnAbs (2, 39) and has been postulated to have as correlates of protection antibody dependent cellular cytotoxicity (ADCC)-mediating antibodies (37, 40-42) and V3 antibodies (43). The present study reaffirms that tier 1 virus-neutralizing antibodies would be of limited benefit in protection from infection against heterologous tier 2 viruses. However, in our study we show that such antibodies could neutralize autologous tier 1B and tier 2 HIV-1 Envs with which they co-evolved (FIG. 4; FIGS. 8 and 9) with which they co-evolved. It is important to note that there is one clinical setting where restricted tier 1 autologous virus-neutralizing antibodies could be potentially protective—that of mother-to-child transmission (MTCT) (44). Maternal IgG antibodies are actively transferred to the developing fetus over the second half of gestation (45), and the presence of maternally-derived antibodies could plausibly prevent newborn infection. Thus, V3- or CD4bs-directed antibodies of the type described here could correlate with decreased transmission risk for MTCT. In a companion paper (Permar S R et al.), a study of the correlates of transmission risk in the Women and Infants Transmission Study (WITS) has indeed demonstrated that the correlates of transmission risk are plasma tier 1 virus-neutralizing antibodies. Thus, induction of high levels of V3 and CD4bs autologous neutralizing antibodies by an Env vaccine in pregnant women might be expected to reduce intrapartum and peripartum HIV-1 transmission to infants that occurs in mothers that arrive late to antenatal care or despite peripartum treatment with anti-retroviral drugs (46).

Materials and Methods:

The clinical material used for the present study was obtained as a part of the CHAVI 001 observational study. The participants studied here were identified during the screening of CHAVI 001 and CHAVI 008 subjects for the presence of neutralization breadth (47). The present work was performed under a protocol approved by the Duke University Health System Institutional Review Board for Clinical Investigations. These original studies with human subjects from which we obtained the clinical material herein studied were approved by the Kilimanjaro Christian Medical Centre Research Ethics Committee, the Tanzania National Institutes for Medical Research Ethics Coordinating Committee, and the Institutional Review Boards of the London School of Hygiene and Tropical Medicine and Duke University as well as by the NIH Human Subject Review Committee.

Clinical Material.

The participants in this study (CH0457 and CH505) were recruited in 2008 in Tanzania and Malawi, respectively. At the time of recruitment, CH0457 had been chronically infected with a subtype C virus for an unknown period. This participant did not receive antiretroviral drug therapy during the study period. Peripheral blood collections were performed at weeks 0, 2, 4, 8, 12, 16, 24, 48, 72, and 96 of observation. Blood was processed for peripheral blood mononuclear cells (PBMC), plasma, and serum, all of which were cryopreserved for transport to the research laboratories. Participant CH505 was recruited early following infection and has been described previously (16).

Flow Cytometry Panel Antibodies, Recombinant Proteins, and Assay Control Antibodies.

The gp120_{ConC} core protein was produced as described (48) and labeled with Pacific Blue and Alexa Fluor (AF) 647 using fluorochrome labeling kits (Invitrogen, Carlsbad, Calif.). The protein batches were confirmed to bind to CD4 expressed on the surface of the H9 T cell line as a quality control after conjugation. Setup for flow cytometry was performed as described (49). Sorting was performed using antibodies reactive with surface IgM (FITC), surface IgD (phycoerythrin [PE]), CD3 (PE-Cy5), CD16 (PE-Cy5), CD235a (PE-Cy5), and CD19 (allophycocyanin [APC]-Cy7) (BD Biosciences, San Jose, Calif.); CD14 (PE-Cy5) (Invitrogen, Carlsbad, Calif.); CD27 (PE-Cy7) and CD38 (APC-Alexa Fluor 700) (Beckman Coulter, Brea, Calif.).

Hyperimmune HIV-1 globulin subtype C (HIVIG-C) is a mixture of purified IgG from 5 subtype C HIV-1-infected plasma donors in South Africa (Johannesburg blood bank). (50). Genetic subtype was confirmed by SGA sequencing of the plasma Envs. The 5 IgG samples included in HIVIG-C were selected among 35 IgG samples for having the greatest magnitude and breadth of neutralizing activity against a panel of 6 tier 2 viruses. Palivizumab, a humanized monoclonal antibody against the F protein of respiratory syncytial virus, was purchased from MedImmune, LLC (Gaithersburg, Md.). Negative control CH65 is a mAb directed against the sialic acid binding site of hemagglutinin (51, 52). Positive control CH31 is a bnAb directed against the CD4bs (29, 53), as is positive control CH106 (16). Positive control was CD4bs-directed BNAb HJ16 (25).

Antibody Reactivity by Binding Antibody Multiplex Assay and Enzyme-Linked Immunosorbant Assay (ELISA).

Expressed mAbs were studied for reactivity to HIV-1 antigens using a standardized custom binding antibody multiplex assay using Luminex (54). All assays were run under conditions compliant with Good Clinical Laboratory Practice, including tracking of positive controls by Levy-Jennings charts. FDA-compliant software, Bio-Plex Manager, version 5.0 (Bio-Rad, Hercules, Calif.), was utilized for the analysis of specimens. Screening by binding antibody multiplex assays was performed against a panel of HIV-1 antigens (gp140_{ConC}, gp120_{ConC} full length, gp140_{ConB}, gp140_{ConG}, gp140_{JR.FL}); mAbs that had a blank-bead-subtracted value greater than 2000 units and greater than 1000 times the mAb IgG concentration in g/mL were evaluated further. Binding of all mAbs was confirmed by subsequent assays on mAbs prepared from transfected cells at large scales.

ELISA testing of mAbs was performed as described (55); testing was considered positive if the optical density reading at 405 nm was above 0.3 units and greater than 4-fold over background.

Flow Cytometric Analysis and Single-Cell Sorting.

We previously reported that CH0457 had broad neutralizing activity in plasma that could be absorbed by a subtype C consensus (ConC) gp120 protein that lacked V1V2 and V3 loops (gp120_{ConC} core) (47). To isolate neutralizing antibody-producing memory B cells, we used antigen-specific sorting. Fluorescently-labeled gp120_{ConC} core protein was used to isolate Env-reactive memory B cells using a dual-color technique (13, 56). We sorted samples from the week 8 and week 12 time points, and in both cases we isolated antigen-specific B cells from which immunoglobulin (Ig) genes were recovered (Fig. S1 online). In total, we isolated 19 heavy chains with paired light chains and found that when expressed as mAbs, 12/19 (63%) were reactive with one or more consensus Env proteins from clades A, B, C, G and CRF01_AE; 11 of these mAbs were carried forward for further study (Table S1).

Single-cell sorting was performed using a BD FACSAria II (BD Biosciences, San Jose, Calif.) and the flow cytometry data were analyzed using FlowJo (Treestar, Ashland, Oreg.). Antigen-specific memory B cells were identified by using gp120_{ConC} core labeled with Alexa Fluor 647 and Pacific Blue; cells were gated on CD3− CD14− CD16− CD235a− CD19+ surface IgD-gp120_{ConC} core+/+. Single cells were directly sorted into 96-well plates containing 20 μL per well of reverse transcription (RT) reaction buffer (5 μL of 5′ first-strand cDNA buffer, 0.5 μL of RNaseOUT [Invitrogen, Carlsbad, Calif.], 1.25 μL of dithiothreitol, 0.0625 μL Igepal CA-630 [Sigma, St. Louis, Mo.], 13.25 μL of distilled H₂O [dH₂O; Invitrogen, Carlsbad, Calif.]); plates were stored at −80° C. until use and after sorting were again stored at −80° C. until PCR was performed.

PCR Isolation and Analysis of Immunoglobulin (Ig) V_{H}, V_{κ}, and V_{λ} Genes.

Single-cell PCR was performed as described (49, 57, 58). PCR amplicons were sequenced in forward and reverse directions using a BigDye sequencing kit on an ABI 3730XL (Applied Biosystems, Foster City, Calif.). Sequence base calling was performed using Phred (59, 60), forward and reverse strands were assembled using an algorithm based on the quality scores at each position (61). Local alignment with known sequences was used to determine Ig isotype (62); V, D, and J region genes, complementarity-determining region 3 (CDR3) lengths, and mutation frequencies were determined using SoDA (63). Clonal lineages of antibodies were determined as described (51, 56) and were confirmed by alignment of complete V(D)J sequences. Maximum-likelihood trees for clonal lineages were generated using V(D)J regions (excluding constant region sequences); trees were constructed (dnaml), reorganized (retree), and plotted (drawgram) with the PHYLIP package, version 3.69 (64).

Expression of V_{H} and V_{κ/λ} as Full-Length IgG1 mAbs.

PCR was used to assemble isolated Ig V_{H} and V_{κ/λ} gene pairs into linear full-length Ig heavy- and light-chain gene expression cassettes as described (57). Human embryonic kidney cell line 293T (ATCC, Manassas, Va.) was grown to near confluence in six-well tissue culture plates (Becton Dickinson, Franklin Lakes, N.J.) and transfected with 2 μg per well of both IgH and Igκ/λ purified PCR-produced cassettes using Effectene (Qiagen, Valencia, Calif.). Culture supernatants were harvested 3 days after transfection and concentrated 4-fold using centrifugal concentrators; expressed IgG was quantitated by ELISA (65); tested mAbs were expressed at 10 μg/mL up to 20 mg/mL. Larger-scale production of mAbs was performed using synthesized linear IgH and Igκ/λ gene constructs (GeneScript, Piscataway, N.J.).

Amplification of Full-Length Env Genes.

Viral RNA (vRNA) was prepared from plasma samples (400 μL) using the EZ1 Virus Mini Kit V2.0 on BIO ROBOT EZ1 (Qiagen; Valencia, Calif.). Reverse transcription was performed with 20 μL of vRNA and 80 pmol primer 1.R3.B3R (5′-ACTACTTGAAGCACTCAAGGCAAGCTTTATTG-3′) in 50 μL using Superscript III (Invitrogen; Carlsbad, Calif.). The 3′ half genomes were amplified by single genome amplication (SGA) as previous described (66, 67), using 07For7 (5′CAAATTAYAAAAATTCAAAATTTTCGGGTTTATTACAG-3′) and 2.R3.B6R (5′-TGAAGCACTCAAGGCAAGCTTTATTGAGGC-3′) as first round primers, and VIF1 (5′-GGGTTTATTACAGGGACAGCAGAG-3′) and Low2c (5′-TGAGGCTTAAGCAGTGGGTTCC-3′) as the second round primers. The PCR products were purified with the QiaQuick PCR Purification kit (Qiagen; Valencia, Calif.). The env gene sequences were obtained by cycle-sequencing and dye terminator methods with an ABI 3730XL genetic analyzer (Applied Biosystems; Foster City, Calif.). Individual sequence contigs from each env SGA were assembled and edited using the Sequencher program 4.7 (Gene Codes; Ann Arbor, Mich.).

Amplification of HIV-1 Env Genes from PBMCs by SGA.

Proviral DNA was extracted from 3×10⁶ PBMCs at the enrollment (week 0) time point using the QIAamp DNA Blood and Tissue kit (Qiagen; Valencia, Calif.). The HIV-1 rev/env cassette was amplified from the genomic DNA using the single genome amplification (SGA) method. The PCR primers and conditions were the same as those used for viral RNA templates extracted from plasma.

Generation of Pseudoviruses.

The CMV promoter was added to the 5′ end of each env gene amplified by SGA using the promoter addition PCR (pPCR) method as described (68). The pPCR product was used for generation of pseudoviruses by cotransfecting with the env-deficient HIV-1 backbone pSG3Δenv into 293T cells in a 6-well tissue culture plate using FuGENE6 transfection reagent (Roche Diagnostics; Indianapolis, Ind.) according to manufacturer instructions. Transfected cells were maintained in DMEM with 10% FBS at 37° C. with 5% CO₂. Forty-eight hours after transfection, supernatants were harvested and stored in 20% FBS medium at −80° C.

Neutralization Assay in TZM-bl Cells.

Neutralizing antibody assays in TZM-bl cells were performed as described (69). Antibodies were tested at concentrations up to 50 μg/mL using eight serial 3-fold dilutions. Control antibodies include HJ16 which was generously provided by D. Corti (Institute for Research in Biomedicine, Universita della Svizzera Italiana, Bellinzona, Switzerland). Env-pseudotyped viruses were added to the antibody dilutions at a predetermined titer to produce measurable infection and incubated for 1 h. TZM-bl cells were added and incubated for 48 h. Firefly luciferase (Luc) activity was measured as a function of relative luminescence units (RLU) using a Britelite Luminescence Reporter Gene Assay System as described by the supplier (Perkin-Elmer Life Sciences, Waltham, Mass.). Neutralization was calculated as the reduction in RLU in test wells compared with control wells after subtraction of background RLU in cell control wells and reported as mAb 50% inhibitory concentration (IC50) in μg/mL. Env-pseudotyped viruses were prepared in 293T cells and titrated in TZM-bl cells as described (69).

Mapping of mAb Specificities by Neutralization.

Single amino acid substitutions were introduced into the consensus C (ConC) or B.RHPA Env by oligonucleotide-directed PCR mutagenesis using the QuickChange site-directed mutagenesis kit (Stratagene, La Jolla, Calif.). Alanine or conserved mutations were introduced in C1 (L125A), V1 (R132A/T), C2 (S256A, N289K), C3 (T372V, T373M, S375M), C5 (G471E), the β23 sheet of C4 (R456W), as well as the CD4bs (D-loop: N276A/Q, T278A, N279D and α5: D474A, M475A, R476A). The ability of antibodies to neutralize pseudoviruses containing Env point mutations was assessed and compared to the wild-type pseudovirus neutralization. A fifteen-fold or higher increase in IC₅₀ titer from the wild-type to the mutant was considered positive.

Statistical Analysis.

Graphs of the data were created using GraphPad Prism (GraphPad Software, La Jolla, Calif.) with layout in Illustrator CS5 (Adobe, San Jose, Calif.). Statistical tests were performed in SAS, version 9.2 (SAS Institute, Cary, N.C.) or in R, version 2.15.2 (R Foundation for Statistical Computing, Vienna, Austria). The statistical test used is noted when p values are presented. Env sequence phylogenies were inferred using PhyML (70) with the HIVw substitution model (71).

### REFERENCES

1. J. R. Mascola, B. F. Haynes, HIV-1 neutralizing antibodies: understanding nature's pathways. Immunol Rev 254, 225-244 (2013).
2. D. C. Montefiori et al., Magnitude and breadth of the neutralizing antibody response in the RV144 and Vax003 HIV-1 vaccine efficacy trials. Journal of Infectious Diseases 206, 431-441 (2012).
3. B. F. Keele et al., Identification and characterization of transmitted and early founder virus envelopes in primary HIV-1 infection. Proceedings of the National Academy of Sciences 105, 7552-7557 (2008).
4. J. R. Mascola, D. C. Montefiori, The role of antibodies in HIV vaccines. Annu Rev Immunol 28, 413-444 (2010).
5. P. Gilbert et al., Magnitude and breadth of a nonprotective neutralizing antibody response in an efficacy trial of a candidate HIV-1 gp120 vaccine. Journal of Infectious Diseases 202, 595-605 (2010).
6. J. Albert et al., Rapid development of isolate-specific neutralizing antibodies after primary HIV-1 infection and consequent emergence of virus variants which resist neutralization by autologous sera. AIDS 4, 107-112 (1990).
7. T. Igarashi et al., Emergence of a highly pathogenic simian/human immunodeficiency virus in a rhesus macaque treated with anti-CD8 mAb during a primary infection with a nonpathogenic virus. Proc Natl Acad Sci USA 96, 14049-14054 (1999).
8. P. L. Moore et al., The c3-v4 region is a major target of autologous neutralizing antibodies in human immunodeficiency virus type 1 subtype C infection. J Virol 82, 1860-1869 (2008).
9. M. E. Laird, T. Igarashi, M. A. Martin, R. C. Desrosiers, Importance of the V1/V2 loop region of simian-human immunodeficiency virus envelope glycoprotein gp120 in determining the strain specificity of the neutralizing antibody response. J Virol 82, 11054-11065 (2008).
10. P. L. Moore, E. S. Gray, L. Morris, Specificity of the autologous neutralizing antibody response. Curr Opin HIV AIDS 4, 358-363 (2009).
11. H. Tang et al., Epitopes immediately below the base of the V3 loop of gp120 as targets for the initial autologous neutralizing antibody response in two HIV-1 subtype B-infected individuals. J Virol 85, 9286-9299 (2011).
12. K. J. Bar et al., A. Trkola, Ed. Early low-titer neutralizing antibodies impede HIV-1 replication and select for virus escape. PLoS Pathog 8, e1002721 (2012).
13. E. S. Gray et al., Isolation of a monoclonal antibody that targets the alpha-2 helix of gp120 and represents the initial autologous neutralizing-antibody response in an HIV-1 subtype C-infected individual. J Virol 85, 7719-7729 (2011).
14. P. L. Moore et al., Evolution of an HIV glycan-dependent broadly neutralizing antibody epitope through immune escape. Nat Med 18, 1688-1692 (2012).
15. P. L. Moore et al., Multiple pathways of escape from HIV broadly cross-neutralizing V2-dependent antibodies. J Virol 87, 4882-4894 (2013).
16. H.-X. Liao et al., Co-evolution of a broadly neutralizing HIV-1 antibody and founder virus. Nature 496, 469-476 (2013).
17. C. K. Wibmer et al., A. Trkola, Ed. Viral escape from HIV-1 neutralizing antibodies drives increased plasma neutralization breadth through sequential recognition of multiple epitopes and immunotypes. PLoS Pathog 9, e1003738 (2013).
18. P. Hraber et al., Prevalence of broadly neutralizing antibody responses during chronic HIV-1 infection. AIDS 28, 163-169 (2014).
19. J. Swetnam, E. Shmelkov, S. Zolla-Pazner, T. Cardozo, Comparative magnitude of cross-strain conservation of HIV variable loop neutralization epitopes. PLoS ONE 5, e15994 (2010).
20. M. K. Gorny et al., Preferential use of the VH5-51 gene segment by the human immune response to code for antibodies against the V3 domain of HIV-1. Mol Immunol 46, 917-926 (2009).
21. M. K. Gorny et al., Human monoclonal antibodies to the V3 loop of HIV-1 with intra- and interclade cross-reactivity. J Immunol 159, 5114-5122 (1997).
22. S. A. Jeffs et al., Characterization of human monoclonal antibodies selected with a hypervariable loop-deleted recombinant HIV-1(IIIB) gp120. Immunol. Lett. 79, 209-213 (2001).
23. J. P. Moore, Q. J. Sattentau, R. Wyatt, J. Sodroski, Probing the structure of the human immunodeficiency virus surface glycoprotein gp120 with a panel of monoclonal antibodies. J Virol 68, 469-484 (1994).
24. R. Pantophlet, T. Wrin, L. A. Cavacini, J. E. Robinson, D. R. Burton, Neutralizing activity of antibodies to the V3 loop region of HIV-1 gp120 relative to their epitope fine specificity. Virology 381, 251-260 (2008).
25. D. Corti et al., D. Unutmaz, Ed. Analysis of Memory B Cell Responses and Isolation of Novel Monoclonal Antibodies with Neutralizing Breadth from HIV-1-Infected Individuals. PLoS ONE 5, e8805 (2010).
26. S. S. Balla-Jhagjhoorsingh et al., C. M. Gray, Ed. The N276 glycosylation site is required for HIV-1 neutralization by the CD4 binding site specific HJ16 monoclonal antibody. PLoS ONE 8, e68863 (2013).
27. D. S. Ruelas, W. C. Greene, An integrated overview of HIV-1 latency. Cell 155, 519-529 (2013).
28. F. Gao et al., Cooperation of B Cell Lineages in Induction of HIV-1-Broadly Neutralizing Antibodies. Cell 158, 481-491 (2014).
29. X. Wu et al., Focused evolution of HIV-1 neutralizing antibodies revealed by structures and deep sequencing. Science 333, 1593-1602 (2011).
30. M. S. Seaman et al., Tiered categorization of a diverse panel of HIV-1 Env pseudoviruses for assessment of neutralizing antibodies. J Virol 84, 1439-1452 (2010).
31. X. Wei et al., Antibody neutralization and escape by HIV-1. Nature 422, 307-312 (2003).
32. D. C. Montefiori et al., Viremia control despite escape from a rapid and potent autologous neutralizing antibody response after therapy cessation in an HIV-1-infected individual. J Immunol 170, 3906-3914 (2003).
33. D. D. Richman, T. Wrin, S. J. Little, C. J. Petropoulos, Rapid evolution of the neutralizing antibody response to HIV type 1 infection. Proceedings of the National Academy of Sciences 100, 4144-4149 (2003).
34. H. Haim et al., A. Trkola, Ed. Contribution of intrinsic reactivity of the HIV-1 envelope glycoproteins to CD4-independent infection and global inhibitor sensitivity. PLoS Pathog 7, e1002101 (2011).
35. H. Haim et al., Modeling virus- and antibody-specific factors to predict human immunodeficiency virus neutralization efficiency. Cell Host Microbe 14, 547-558 (2013).
36. S. D. W. Frost et al., Neutralizing antibody responses drive the evolution of human immunodeficiency virus type 1 envelope during recent HIV infection. Proc Natl Acad Sci USA 102, 18514-18519 (2005).
37. B. F. Haynes et al., Immune-correlates analysis of an HIV-1 vaccine efficacy trial. N Engl J Med 366, 1275-1286 (2012).
38. S. M. Hammer et al., Efficacy trial of a DNA/rAd5 HIV-1 preventive vaccine. N Engl J Med 369, 2083-2092 (2013).
39. S. Rerks-Ngarm et al., Vaccination with ALVAC and AIDSVAX to Prevent HIV-1 Infection in Thailand. N Engl J Med 361, 2209-2220 (2009).
40. M. Bonsignori et al., Antibody-dependent cellular cytotoxicity-mediating antibodies from an HIV-1 vaccine efficacy trial target multiple epitopes and preferentially use the VH1 gene family. J Virol 86, 11521-11532 (2012).
41. G. D. Tomaras et al., Vaccine-induced plasma IgA specific for the C1 region of the HIV-1 envelope blocks binding and effector function of IgG. Proceedings of the National Academy of Sciences 110, 9019-9024 (2013).
42. H.-X. Liao et al., Vaccine induction of antibodies against a structurally heterogeneous site of immune pressure within HIV-1 envelope protein variable regions 1 and 2. Immunity 38, 176-186 (2013).
43. R. Gottardo et al., Z. Chen, Ed. Plasma IgG to linear epitopes in the V2 and V3 regions of HIV-1 gp120 correlate with a reduced risk of infection in the RV144 vaccine efficacy trial. PLoS ONE 8, e75665 (2013).
44. C. Wettstein et al., Missed opportunities to prevent mother-to-child-transmission: systematic review and meta-analysis. AIDS 26, 2361-2373 (2012).
45. A. Malek, R. Sager, P. Kuhn, K. H. Nicolaides, H. Schneider, Evolution of maternofetal transport of immunoglobulins during human pregnancy. Am. J. Reprod. Immunol. 36, 248-255 (1996).
46. C. S. Chasela et al., Maternal or Infant Antiretroviral Drugs to Reduce HIV-1 Transmission. N Engl J Med 362, 2271-2281 (2010).
47. G. D. Tomaras et al., Polyclonal B cell responses to conserved neutralization epitopes in a subset of HIV-1-infected individuals. J Virol 85, 11502-11519 (2011).
48. E. S. Gray et al., The Neutralization Breadth of HIV-1 Develops Incrementally over Four Years and Is Associated with CD4+ T Cell Decline and High Viral Load during Acute Infection. J Virol 85, 4828-4840 (2011).
49. M. A. Moody et al., HIV-1 gp120 vaccine induces affinity maturation in both new and persistent antibody clonal lineages. J Virol 86, 7496-7507 (2012).
50. L. Morris et al., Isolation of a human anti-HIV gp41 membrane proximal region neutralizing antibody by antigen-specific single B cell sorting. PLoS ONE 6, e23532 (2011).
51. M. A. Moody et al., H3N2 influenza infection elicits more cross-reactive and less clonally expanded anti-hemagglutinin antibodies than influenza vaccination. PLoS ONE 6, e25797 (2011).
52. J. R. R. Whittle et al., Broadly neutralizing human antibody that recognizes the receptor-binding pocket of influenza virus hemagglutinin. Proceedings of the National Academy of Sciences 108, 14216-14221 (2011).
53. M. Bonsignori et al., Two distinct broadly neutralizing antibody specificities of different clonal lineages in a single HIV-1-infected donor: implications for vaccine design. J Virol 86, 4688-4692 (2012).
54. G. D. Tomaras et al., Initial B-cell responses to transmitted human immunodeficiency virus type 1: virion-binding immunoglobulin M (IgM) and IgG antibodies followed by plasma anti-gp41 antibodies with ineffective control of initial viremia. J Virol 82, 12449-12463 (2008).
55. S. M. Alam et al., Human immunodeficiency virus type 1 gp41 antibodies that mask membrane proximal region epitopes: antibody binding kinetics, induction, and potential for regulation in acute infection. J Virol 82, 115-125 (2008).
56. M. Moody et al., Antibody lineages with evidence of somatic hypermutation persisting for >4 years in a South African subject with broad neutralizing activity. Retrovirology 9, P85 (2012).
57. H.-X. Liao et al., High-throughput isolation of immunoglobulin genes from single human B cells and expression as monoclonal antibodies. Journal of Virological Methods 158, 171-179 (2009).
58. J. Wrammert et al., Rapid cloning of high-affinity human monoclonal antibodies against influenza virus. Nature 453, 667-671 (2008).
59. B. Ewing, L. Hillier, M. C. Wendl, P. Green, Base-calling of automated sequencer traces using phred. I. Accuracy assessment. Genome Res. 8, 175-185 (1998).
60. B. Ewing, P. Green, Base-calling of automated sequencer traces using phred. II. Error probabilities. Genome Res. 8, 186-194 (1998).
61. T. B. Kepler et al., Chiropteran types I and II interferon genes inferred from genome sequencing traces by a statistical gene-family assembler. BMC Genomics 11, 444 (2010).
62. T. F. Smith, M. S. Waterman, Identification of common molecular subsequences. J Mol Biol 147, 195-197 (1981).
63. J. M. Volpe, L. G. Cowell, T. B. Kepler, SoDA: implementation of a 3D alignment algorithm for inference of antigen receptor recombinations. Bioinformatics 22, 438-444 (2006).
64. J. Felsenstein, PHYLIP (Phylogeny Inference Package) (2009).
65. E. S. Gray et al., Antibody specificities associated with neutralization breadth in plasma from human immunodeficiency virus type 1 subtype C-infected blood donors. J Virol 83, 8925-8937 (2009).
66. J. F. Salazar-Gonzalez et al., Genetic identity, biological phenotype, and evolutionary pathways of transmitted/founder viruses in acute and early HIV-1 infection. J Exp Med 206, 1273-1289 (2009).
67. C. Jiang et al., Primary infection by a human immunodeficiency virus with atypical coreceptor tropism. J Virol 85, 10669-10681 (2011).
68. J. L. Kirchherr et al., High throughput functional analysis of HIV-1 env genes without cloning. Journal of Virological Methods 143, 104-111 (2007).
69. D. C. Montefiori, Evaluating neutralizing antibodies against HIV, SIV, and SHIV in luciferase reporter gene assays. Curr Protoc Immunol Chapter 12, Unit 12.11 (2005).
70. S. Guindon, O. Gascuel, A simple, fast, and accurate algorithm to estimate large phylogenies by maximum likelihood. Syst. Biol. 52, 696-704 (2003).
71. D. C. Nickle et al., O. Pybus, Ed. HIV-specific probabilistic models of protein evolution. PLoS ONE 2, e503 (2007).
72. P. D. Kwong et al., Structures of HIV-1 gp120 envelope glycoproteins from laboratory-adapted and primary isolates. Structure 8, 1329-1339 (2000).
73. S. S. Balla-Jhagjhoorsingh et al., L. N. F. Poh, Ed. Characterization of neutralizing profiles in HIV-1 infected patients from whom the HJ16, HGN194 and HK20 mAbs were obtained. PLoS ONE 6, e25488 (2011).

Isolation of nAbs.

Antibodies from CH0457 were isolated by antigen-specific B cell sorting using a clade C consensus Env protein. Clonal lineage CH13 consisted of six monoclonal antibodies (mAbs) of IgG1 isotype (CH13, CH16, CH17, CH18, CH45, CH46) that used V_{H}1˜69*01/J_{H}3*02 and V_{K}1-39*01/J_{K}4*01 genes. Epitope mapping with binding and neutralization assays demonstrated that the CH13 lineage antibody bound to the CD4bs and were sensitive to mutations at D386, E370, I371, S375, and K421 (FIG. 1c; Tables S2 and S3). Two additional mAbs, CH14 and CH48, were not clonally related to any other mAbs isolated nor to each other, and both mAbs mapped in Env peptide binding assays to the HIV-1 Env third variable (V3) loop (FIG. 1d; Table 9). Like clonal lineage CH13, mAbs CH14 and CH48 neutralized only tier 1 but not tier 2 heterologous HIV-1 strains (FIG. 2).

The second group of mAbs, clonal lineage CH27 (FIG. 1b), consisted of three mAbs that used V_{H}3˜66*02/J_{H}2*01 and V_{K}3˜20*01/J_{K}1*01 (CH27, CH28, CH44). Two members of this clonal lineage (CH27 and CH28) were found to be isotype IgA2 while the third was IgG1 (Table S1). All were expressed as IgG1 mAbs. Testing of this group of mAbs using HIV-1 strain B.RHPA mutants demonstrated that they were sensitive to changes at N276 and T278, suggesting that the CH27 lineage consisted of HJ16-like CD4bs-directed bnAbs (1) (Table S5). Surface plasmon resonance studies of mAbs from the CH27 lineage and HJ16 showed that they cross-blocked each other for binding to HIV-1 Env (FIG. 6).

Plasma samples from CH0457 taken from weeks 8 and 96 were tested against the same panel of heterologous viruses (FIG. 2). Neutralization titers against heterologous viruses were similar at the two chronic infection time points, despite the fact that the samples were collected nearly two years apart. Plasma antibodies neutralized all tier 1 isolates, consistent with the clonal lineage CH13 mAbs and V3 mAbs CH14 and CH48 neutralization patterns. Of the 10 heterologous HIV isolates neutralized by plasma at >1:1000 dilution, nine viruses were neutralized by lineage CH27 mAbs at <2 μg/mL (FIG. 2). Thus, the isolated mAbs accounted for the majority of CH0457 plasma heterologous virus neutralization.

We isolated restricted V3 neutralizing antibodies from a second HIV-1-infected African individual, CH505, 41 weeks after transmission (2). This individual eventually developed a CD4bs clonal lineage (termed CH103) at 136 weeks after transmission (2).

Validation of CH0457 Sequence Integrity.

To determine if there was any evidence for multiple infection or contamination, particularly given that there were two distinctive clades in the CH0457 sample, we did the following tests using the tools at the Los Alamos HIV database (www.hiv.lanl.gov). First we made a DNA consensus of the sequences from the persistent minor clade and the major lineage in CH0457. We then then used HIV-BLAST to these compare the two consensus sequences against the HIV database. Both consensus sequences are closest to natural sequences from CH0457 in already GenBank, supporting that they came from the same quasispecies, and same individual. At the DNA level, the consensus from the persistent minor clade shared between 94 and 97% identity in Blast searches with other CH0457 sequences from the cominant clade. In contrast, the next closest match shared only 87%; it was a C clade isolate from Malawi. We then extracted all full length Env sequences from Tanzania; there were 388 of them. We combined these with the HIV subtyping reference set, and the consensus sequences from CH0457, and made a neighbor joining phylogeny based on these 435 reference and Tanzanian sequences. The two consensus sequences from the 2 distinctive within-subject CH0457 lineages always clustered together, among natural sequences from CH0457, forming a monophyletic group with high bootstrap support in a neighbor joining tree (data not shown, as this was a quality control check). This again indicates that the unusual clade is not a recurrent contamination, or independent infecting strain, and that both lineages evolved from a single infecting strain within CH0457, and had diverged prior to the first sample in taken during chronic infection.

This view is was further supported by the addition of the PBMC proviral DNA sequences from the enrollment time point, that were considered to be biologically “archived” in the host representing virus that had been replicating prior to the time of enrollment. These sequences revealed intermediate steps between the two distinctive lineages found in the CH0457 SGA sequences (FIG. 7). Among the proviral sequences, there were 6 that were highly significantly enriched for G-to-A hypermutated in Apobec motifs (Hypermut, www.hiv.lanl.gov) (3, 4) giving rise to multiple stop codons in Env resulting in clearly inactive virus. These are evident as a hypermutated cluster in the fully phylogenetic tree shown in FIG. 7 (w0.41c, w0.40c, w0.19c, w0.c, w0.13c, w0.48c; highlighted by an asterisk). There were no other significantly hypermutated sequences in the proviral set, and none among the SGA viral sequences.

TABLE S1
HIV-1 Env-reactive antibodies isolated from CH0457.

| | | | heavy chain | | | | light chain | | | |
|---|---|---|---|---|---|---|---|---|---|---|
| | | | | | CDR3 | mutation | | | CDR3 | mutation |
| | week | isotype | V_{H} | J_{H} | length | frequency | V | J | length | frequency |
| non-lineage | | | | | | | | | | |
| CH14 | 12 | IgG1 | 1~69*04 | 3*02 | 17 | 14.8% | κ 4~1*01 | 3*01 | 9 | 8.2% |
| CH48 | 12 | IgG1 | 4~30-4*01 | 4-02 | 19 | 9.5% | λ 2~14*03 | 3*02 | 9 | 6.2% |
| Lineage CH13 | | | | | | | | | | |
| CH13 | 8 | IgG1 | 1~69*01 | 4*01 | 17 | 9.1% | κ 1~39*01 | 4*01 | 9 | 4.0% |
| CH16 | 12 | IgG1 | 1~69*01 | 4*01 | 17 | 12.9% | κ 1~39*01 | 4*01 | 9 | 9.0% |
| CH17 | 12 | IgG1 | 1~69*01 | 4*01 | 17 | 9.9% | κ 1~39*01 | 4*01 | 9 | 5.3% |
| CH18 | 12 | IgG1 | 1~69*01 | 4*01 | 17 | 9.4% | κ 1~39*01 | 4*01 | 9 | 4.3% |
| CH45 | 8 | IgG1 | 1~69*01 | 4*01 | 17 | 8.3% | κ 1~39*01 | 4*01 | 9 | 9.6% |
| CH46 | 8 | IgG1 | 1~69*01 | 4*01 | 17 | 9.1% | κ 1~39*01 | 4*01 | 9 | 8.7% |
| average | | | | | | 9.8% | | | | 6.8% |
| Lineage CH27 | | | | | | | | | | |
| CH27 | 8 | IgA2 | 3~66*02 | 2*01 | 10 | 15.3% | κ 3~20*01 | 1*01 | 11 | 15.6% |
| CH28 | 12 | IgA2 | 3~66*02 | 2*01 | 10 | 14.0% | κ 3~20*01 | 1*01 | 11 | 15.6% |
| CH44 | 8 | IgG1 | 3~66*02 | 2*01 | 10 | 17.7% | κ 3~20*01 | 1*01 | 11 | 16.5% |
| average | | | | | | 15.7% | | | | 15.9% |

TABLE S2
Mapping of mAbs by binding to gp 120 mutants.

| | mAb binding assay to gp 120* | | | | | | | |
|---|---|---|---|---|---|---|---|---|
| | B.HXBc2^{†} | | | | B.YU2 | | | |
| Lineage CH13 | E370K | K421A | R476A | D477A | D368A | E370A | I371A | S375W |
| CH13 | 0.04 | 0.31 | 0.79 | 1.08 | 0.18 | 0.23 | 0.31 | 0.29 |
| CH16 | 0.27 | 0.73 | 1.34 | 1.10 | 0.79 | 0.48 | 0.71 | 0.41 |
| CH17 | 0.07 | 0.68 | 0.91 | 1.23 | 0.78 | 0.46 | 0.60 | 0.37 |

*Data normalized vs. binding to wild type gp120 protein.
^{†}Additional mutants tested for which no binding change was observed: B.HXBc2 K429E, D474V, M475S; B.YU2 G473A, M475A, ΔV1/V2/V3.
^{‡}NR = not reactive to B.HXB2c or B.YU2 gp120 proteins.
Lineage members CH18, CH45, and CH46 not tested.

TABLE S3
Mapping of mAbs by neutralization of clade C consensus variants.

| | Neutralization* | | | | |
|---|---|---|---|---|---|
| | clade C consensus^{†} | | | | |
| | | | T372V | | |
| | R132A | R132T | T373M | S375M | D474A |
| Lineage CH13 | | | | | |
| CH13 | >100 | 1.8 | >50 | >100 | 16 |
| CH16 | 0.5 | 0.5 | 7.3 | >32 | 1.3 |
| CH17 | 91 | >55 | 19 | >100 | 10 |
| CH18 | 0.4 | >15 | >9 | >15 | 2 |
| CH45 | >20 | >20 | 9 | >36 | 8.1 |
| CH46 | — | — | — | — | — |
| Lineage CH27 | | | | | |
| CH27 | 0.7 | 1 | 2.1 | 2.3 | 0.4 |
| CH28 | 0.8 | 0.9 | 2.8 | 1.7 | 0.8 |
| CH44 | 1.5 | 3.2 | 2.5 | 2.6 | 0.6 |

*Data shown is fold increase in concentration required to produce 50% neutralization (increase in IC₅₀ in μg/mL of mAb).
^{†}Other mutants of clade C consensus tested that did not show changes >20 fold for any tested mAb: L125A, S256A, N289K, G471E, M475A, R476A.
^{‡}NR = no neutralization of clade C consensus.
^{§}— = not tested.

TABLE S4
Mapping of V3-directed mAbs from CH0457 by ELISA.

| | EC50* | | | | | | |
|---|---|---|---|---|---|---|---|
| | | | | scaffolded V3 loop | | | |
| non- | | | | antigens | | | |
| line- | V3 loop peptides | | | gp70 | gp70 | Env constructs | |
| age | ConB^{†} | ConC | ConS | B.MN3 | AE.92TH023 | RSC3 | ΔRSC3 |
| CH14 | 0.05 | 0.03 | 0.02 | NB^{‡} | 0.004 | NB | NB |
| CH48 | 0.05 | 0.03 | 0.005 | 1.0 | 6.1 | NB | NB |

*Data shown is half maximal effective concentration (EC₅₀) in μg/mL of mAb.
^{†}ConB = clade B consensus; ConC = clade C consensus; ConS = group M consensus.
^{‡}NB = no binding observed.

TABLE S5
Mapping of lineage CH27 mAbs by neutralization of
B.RHPA mutants.

neutralization*
B.RHPA

| | | | | T278A |
|---|---|---|---|---|
| Lineage CH27 | N160K | N276A | T278A | R456W |
| CH27 | 0.1 | 7.6 | 7.1 | 7 |
| CH28 | 0.3 | >333 | >333 | >307 |
| CH44 | 0.2 | >106 | >106 | >1000 |
| HJ16 | 0.5 | >10 | >10 | >1000 |

*Data shown is fold increase in concentration required to produce 50% neutralization (increase in IC₅₀ in μg/mL of mAb).

TABLE S6
Mapping of V3 mAbs from CH505 by ELISA.

| | EC50* | | | | | | | | |
|---|---|---|---|---|---|---|---|---|---|
| | | | | scaffolded V3 loop antigens | | | | | |
| | V3 loop peptides | | | gp70 | gp70 | gp70 | gp70 | Env constructs | |
| non-lineage | ConB^{†} | ConC | ConS | B.MN3 | AE.92TH023 | ConAG | ConC | RSC3 | ΔRSC3 |
| DH151 | 0.15 | 0.009 | 0.008 | NB^{‡} | 0.003 | 0.002 | 0.002 | NB | NB |
| DH228 | NB | NB | 0.008 | NB | NB | 1.50 | 2.52 | NB | NB |

*Data shown is half maximal effective concentration (EC₅₀) in μg/mL of mAb.
^{†}ConB = clade B consensus; ConC = clade C consensus; ConS = group M consensus.
^{‡}NB = no binding observed.

### REFERENCES

1. S. S. Balla-Jhagjhoorsingh et al., C. M. Gray, Ed. The N276 glycosylation site is required for HIV-1 neutralization by the CD4 binding site specific HJ16 monoclonal antibody. PLoS ONE 8, e68863 (2013).
2. H.-X. Liao et al., Co-evolution of a broadly neutralizing HIV-1 antibody and founder virus. Nature 496, 469-476 (2013).
3. R. S. Harris, M. T. Liddament, Retroviral restriction by APOBEC proteins. Nat Rev Immunol 4, 868-877 (2004).
4. P. P. Rose, B. T. Korber, Detecting hypermutations in viral sequences with an emphasis on G->A hypermutation. Bioinformatics 16, 400-401 (2000).
5. S. Guindon, O. Gascuel, A simple, fast, and accurate algorithm to estimate large phylogenies by maximum likelihood. Syst. Biol. 52, 696-704 (2003).
6. D. C. Nickle et al., O. Pybus, Ed. HIV-specific probabilistic models of protein evolution. PLoS ONE 2, e503 (2007).
7. I. Maljkovic Berry et al., Unequal evolutionary rates in the human immunodeficiency virus type 1 (HIV-1) pandemic: the evolutionary rate of HIV-1 slows down when the epidemic rate increases. J Virol 81, 10625-10635 (2007).
8. I. Maljkovic Berry et al., The evolutionary rate dynamically tracks changes in HIV-1 epidemics: application of a simple method for optimizing the evolutionary rate in phylogenetic trees with longitudinal data. Epidemics 1, 230-239 (2009).
9. M. R. Posner et al., An IgG human monoclonal antibody that reacts with HIV-1/GP120, inhibits virus binding to cells, and neutralizes infection. J Immunol 146, 4325-4332 (1991).
10. D. Corti et al., D. Unutmaz, Ed. Analysis of Memory B Cell Responses and Isolation of Novel Monoclonal Antibodies with Neutralizing Breadth from HIV-1-Infected Individuals. PLoS ONE 5, e8805 (2010).
11. X. Wu et al., Focused evolution of HIV-1 neutralizing antibodies revealed by structures and deep sequencing. Science 333, 1593-1602 (2011).
12. J. R. R. Whittle et al., Broadly neutralizing human antibody that recognizes the receptor-binding pocket of influenza virus hemagglutinin. Proceedings of the National Academy of Sciences 108, 14216-14221 (2011).
13. J. Swetnam, E. Shmelkov, S. Zolla-Pazner, T. Cardozo, Comparative magnitude of cross-strain conservation of HIV variable loop neutralization epitopes. PLoS ONE 5, e15994 (2010).
14. M. K. Gorny et al., Preferential use of the VH5-51 gene segment by the human immune response to code for antibodies against the V3 domain of HIV-1. Mol Immunol 46, 917-926 (2009).
15. M. K. Gorny et al., Human monoclonal antibodies to the V3 loop of HIV-1 with intra- and interclade cross-reactivity. J Immunol 159, 5114-5122 (1997).
16. S. A. Jeffs et al., Characterization of human monoclonal antibodies selected with a hypervariable loop-deleted recombinant HIV-1(IIIB) gp120. Immunol. Lett. 79, 209-213 (2001).
17. J. P. Moore, Q. J. Sattentau, R. Wyatt, J. Sodroski, Probing the structure of the human immunodeficiency virus surface glycoprotein gp120 with a panel of monoclonal antibodies. J Virol 68, 469-484 (1994).
18. D. C. Montefiori et al., Magnitude and breadth of the neutralizing antibody response in the RV144 and Vax003 HIV-1 vaccine efficacy trials. Journal of Infectious Diseases 206, 431-441 (2012).
19. R. Pantophlet, T. Wrin, L. A. Cavacini, J. E. Robinson, D. R. Burton, Neutralizing activity of antibodies to the V3 loop region of HIV-1 gp120 relative to their epitope fine specificity. Virology 381, 251-260 (2008).

### Example 2

### ADCC

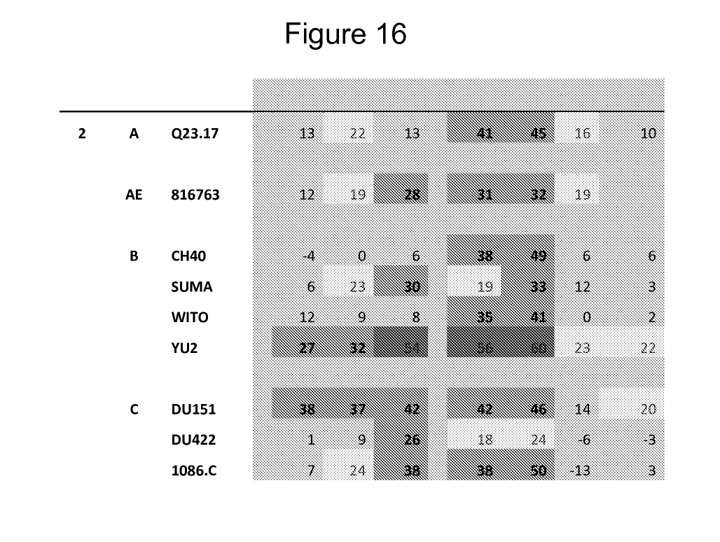

HIV-1 reporter viruses used in ADCC assays were replication-competent infectious molecular clones (IMC) designed to encode the viruses listed in the left column of the Table in FIG. 16, for e.g. SF162.LS (accession number EU123924) or the transmitted/founder WITO.c (accession number JN944948) subtype B env genes in cis within an isogenic backbone that also expresses the Renilla luciferase reporter gene and preserves all viral orfs. The Env-IMC-LucR viruses used were NL-LucR.T2A-SF162.ecto (IMC_{SF162}) and NL-LucR.T2A-WITO.ecto (IMC_{WITO}) (T. G. Edmonds et al., Virology 408, 1 (Dec. 5, 2010)). IMCs were titrated in order to achieve maximum expression within 72 hours post-infection by detection of Luciferase activity and intra-cellular p24 expression. We infected CEM.NKR_{CCR5} cells (NIH AIDS Research and Reference Reagent Repository) with IMC_{SF162} and IMC_{WITO} by incubation with the appropriate TCID₅₀/cell dose of IMC for 0.5 hour at 37° C. and 5% CO₂ in presence of DEAE-Dextran (7.5 μg/ml). The cells were subsequently resuspended at 0.5×10⁶/ml and cultured for 72 hours in complete medium containing 7.5 μg/ml DEAE-Dextran. The infection was monitored by measuring the frequency of cells expressing intracellular p24. Assays performed using the IMC-infected target cells were considered reliable if the percentage of viable p24⁺ target cells was ≧20% on assay day.

A luciferase-based ADCC assay was performed as previously described (H. X. Liao et al., Immunity 38, 176 (Jan. 24, 2013), Pollara J, Bonsignori M, Moody M A, et al. HIV-1 Vaccine-Induced C1 and V2 Env-Specific Antibodies Synergize for Increased Antiviral Activities. J Virol. 2014; 88(14):7715-7726.) Briefly, HIV-1 infected cells, HIV-1 IMC_{SF162} and IMC_{WITO} infected CEM.NKR_{CCR5} cells were used as targets. Whole PBMC obtained from a HIV seronegative donor with the F/V Fc-gamma Receptor (FcRγ) IIIa phenotype were used as the source of NK effector cells. After overnight resting, the PBMC were used as effector cells at an effector to target ratio of 30:1. The target and effector cells were incubated in the presence of 5-fold serial concentrations of plasma/Ab starting at 1:50 dilution for 6 hours at 37° C. in 5% CO₂.] The final read-out was the luminescence intensity generated by the presence of residual intact target cells that have not been lysed by the effector population in presence of ADCC-mediating mAb. The % of killing was calculated using the formula:

$$\% killing = \frac{( RLU of Target + Effector well ) - ( RLU of test well )}{RLU of Target + Effector well} \times 100$$

In this analysis, the RLU of the target plus effector wells represents spontaneous lysis in absence of any source of Ab. Plasma samples collected from a HIV-1 seronegative and seropositive donor were used as negative and positive control samples, respectively, in each assay.

Results are shown in FIG. 16.

All documents and other information sources cited herein are hereby incorporated in their entirety by reference.

What is claimed is:

1. An antibody having the binding specificity of anti-HIV-1 antibody CH27, CH28 or CH 44, or antigen binding fragment thereof.

2. The antibody of claim 1, wherein the HIV-1 antibody is broadly neutralizing.

3. The antibody according to claim 1 wherein the antibody, or fragment thereof, comprises a heavy or light chain amino acid sequence set forth in FIG. 14.

4. The antibody of claim 1, wherein the antibody has the binding specificity of CH44.

5. The antibody according to claim 1 wherein the antibody is an IgA antibody.

6. The antibody according to claim 3 wherein the antibody is CH27 or CH28.

7. An isolated nucleic acid comprising a nucleotide sequence encoding the antibody according to claim 1, or the binding fragment thereof.

8. The nucleic acid according to claim 5 wherein the nucleic acid is present in a vector.

9. A method of preventing or treating HIV-1 comprising administering to a subject in need thereof the antibody, or the fragment thereof, according to claim 1 in an amount sufficient to effect said prevention or treatment.

10. The method according to claim 9, wherein the subject is a human.

11. The method according to claim 9, wherein the antibody is an IgA antibody and the antibody, or the fragment thereof, is administered to a mucosal surface of said subject.

12. A method of preventing or treating HIV-1 comprising administering to a subject in need thereof the nucleic acid according to claim 5 under conditions such that the nucleotide sequence is expressed and the antibody, or fragment thereof, is produced in an amount sufficient to effect the prevention or treatment.

13. A composition comprising the antibody, or fragment thereof, according to claim 1, or the nucleic acid according to claim 5, and a carrier.

14. The composition according to claim 11 wherein the composition is in a form suitable for injection.

15. The composition according to claim 11 wherein the composition is in the form of a cream or ointment.
